(19) United States
(12) Patent Application Publication
(10) Pub. No.: US 2003/0108919 A1
(43) Pub. Date: Jun. 12, 2003

(54) Methods for amplification of nucleic acids

(75) Inventors: Curtis Kautzer, San Jose, CA (US); Nila Patil, Woodside, CA (US); Coleen Hacker, San Carlos, CA (US); David P. McDonough, Cupertino, CA (US); Daryl J. Thomas, Menlo park, CA (US); Wade A. Barrett, Santa Clara, CA (US); John B. Sheehan, Mountain View, CA (US)

Correspondence Address: BEYER WEAVER & THOMAS LLP, P.O. BOX 778, BERKELEY, CA, 94704-0778, US

(73) Assignee: Perlegen Sciences, Inc.

(21) Appl. No.: 10/236,480

(22) Filed: Sep. 5, 2002

Related U.S. Application Data
(63) Continuation-in-part of application No. 10/042,406, filed on Jan. 9, 2002.
(63) Continuation-in-part of application No. 10/042,492, filed on Jan. 9, 2002.
(60) Provisional application No. 60/317,311, filed on Sep. 5, 2001.

(51) Int. Cl. C12Q 1/68; G06G 7/48; G06G 7/58; G06F 19/00; G01N 33/48; G01N 33/50; C12P 19/34

(52) U.S. Cl. 435/6; 435/91.2; 702/20; 703/11

(57) ABSTRACT

The presently claimed invention provides methods for amplifying a DNA target sequence. One embodiment of the present invention provides robust methods for amplification of target sequences. In a first aspect of the invention, a method for selecting primer pairs for the amplification reaction is provided. In a further aspect of the invention, reagents and cycling parameters for the amplification reaction are provided.

### CROSS REFERENCE TO RELATED APPLICATIONS

This application claims priority to provisional application U.S. Ser. No. 60/317,311 filed Sep. 5, 2001, and to U.S. Ser. No. 10/042,406, filed Jan. 9, 2002 and U.S. Ser. No. 10/042,492, filed Jan. 9, 2002, each of which is incorporated by reference in its entirety for all purposes.

### COPYRIGHT NOTICE

A portion of the disclosure of this patent document contains material which is subject to copyright protection. The copyright owner has no objection to the xerographic reproduction by anyone of the patent document or the patent disclosure exactly as it appears in the Patent and Trademark Office patent file or records, but otherwise reserves all copyrights whatsoever.

### BACKGROUND OF THE INVENTION

The polymerase chain reaction (PCR) is a powerful method for amplifying nucleic acid sequences. Various disclosures involving this technique are found in U.S. Pat. Nos. 4,683,202; 4,683,195; 4,800,159; 4,965,188; and 5,512,462, each of which is incorporated herein by reference. In a simple form, PCR is an in vitro technique for the enzymatic synthesis of specific DNA sequences using two oligonucleotide primers that hybridize to complementary nucleic acid strands and flank a region that is to be amplified in a target DNA. A series of reaction steps of 1) template denaturation, 2) primer annealing, and 3) extension of annealed primers by DNA polymerase, results in the geometric accumulation of a specific fragment whose termini are defined by the 5′ ends of the primers. As is well known, PCR is capable of selective enrichment of specific DNA sequences by a factor of 10⁹.

PCR has been applied widely in molecular biology for sequencing, genome mapping and forensics. However, despite such wide-spread use, amplifying long stretches of DNA, particularly genomic DNA, is difficult. Many protocols for long range PCR exist; however, reaction conditions are usually optimized for amplifying specific target regions of interest. Applying the same “optimized” reaction conditions to amplify a different target region may not result in a detectable amplification product.

In light of the above limitations, there is a need in the art for methods capable of amplifying long nucleic acid sequences. The resulting methods may be used in some embodiments to amplify mammalian target sequences across the genome to facilitate genotyping studies, and for other applications in the art of molecular biology.

### SUMMARY OF THE INVENTION

The presently claimed invention provides methods for amplifying a DNA target sequence. One embodiment of the present invention provides robust methods for amplification of target sequences. In a first aspect of the invention, a method for designing primer pairs for the amplification reaction is provided. In a further aspect of the invention, reagents and cycling parameters for the amplification reaction are provided.

Thus, the present invention provides a method for designing primer pairs for amplifying a target sequence, comprising the steps of: choosing a reference sequence; removing at least selected repeat regions in the reference sequence to yield removed and unremoved reference sequence; selecting primer sequences from the unremoved reference sequence according to two or more parameters including primer length and primer melting temperature to yield a set of primers; evaluating the set of primers for extent of coverage and overlap of the reference sequence; and selecting a subset of primer pairs having minimal overlap from the set of primers.

In addition, the present invention provides a method for amplifying a target sequence, comprising the steps of: mixing a reaction cocktail comprising deoxynucleotide triphosphates, target DNA, a divalent cation, DNA polymerase enzyme, a broad spectrum solvent, a zwitterionic buffer and at least one primer pair designed by the method above; heating the reaction cocktail at a denaturing temperature of about 90.0° C. to about 96.0° C. for about 1.0 second to about 30.0 seconds; cooling the reaction cocktail at an annealing/extension temperature of about 50.0° C. to about 68.0° C. for about 1.0 minute to about 28.0 minutes; repeating the heating and cooling steps at least about 10 times; and cooling the reaction cocktail to 4.0° C. in a final cooling step.

Other and further objects, features and advantages would be apparent and eventually more readily understood by reading the following specification and by reference to the accompanying drawings forming a part thereof, or any examples of the presently preferred embodiments of the invention given for the purpose of the disclosure.

### DETAILED DESCRIPTION OF THE FIGURES

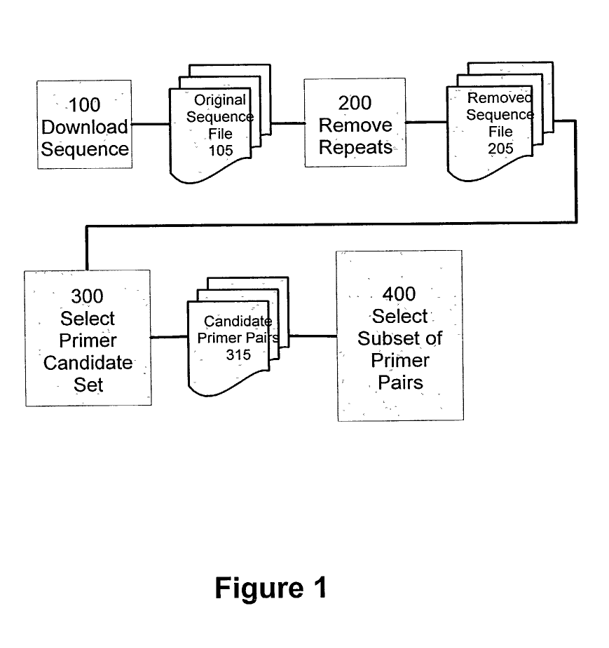

FIG. 1 is a flow chart showing the primer pair selection process.

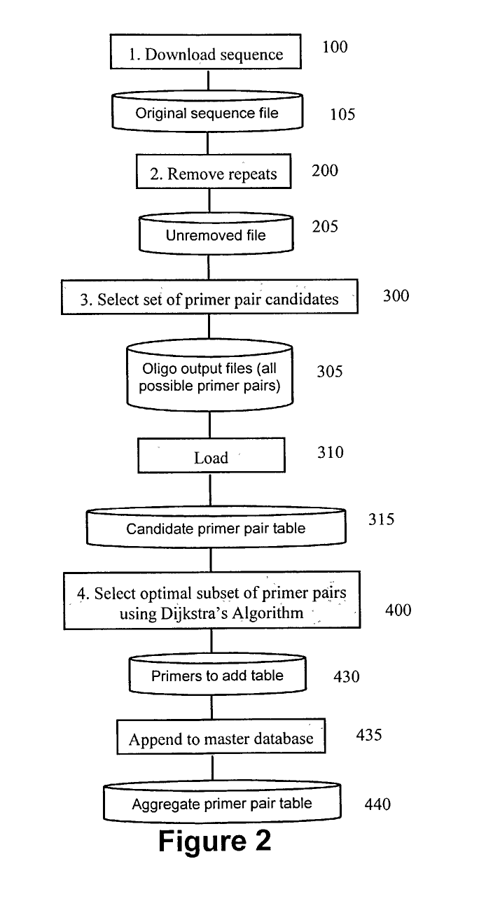

FIG. 2 is a flow chart showing a detailed primer pair selection process according to one embodiment of the present invention.

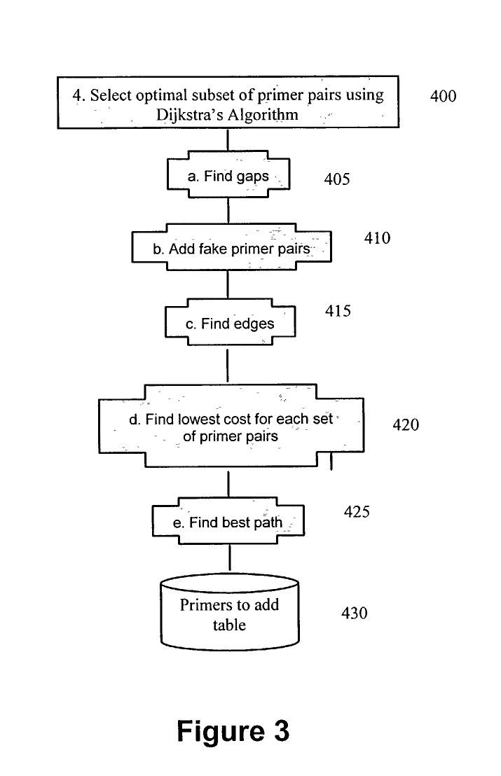

FIG. 3 shows the sub-routines utilized to select the subset of primer pairs in the fourth step of the primer pair selection process.

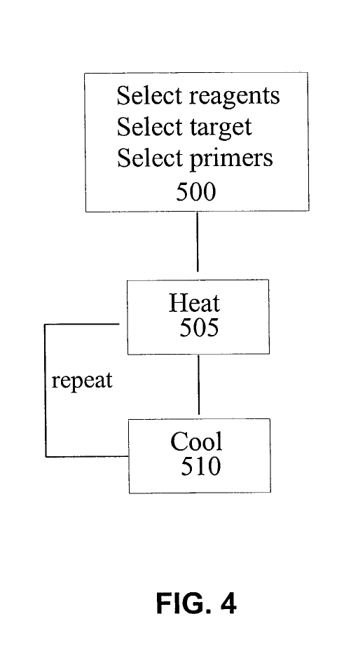

FIG. 4 shows a basic amplification process.

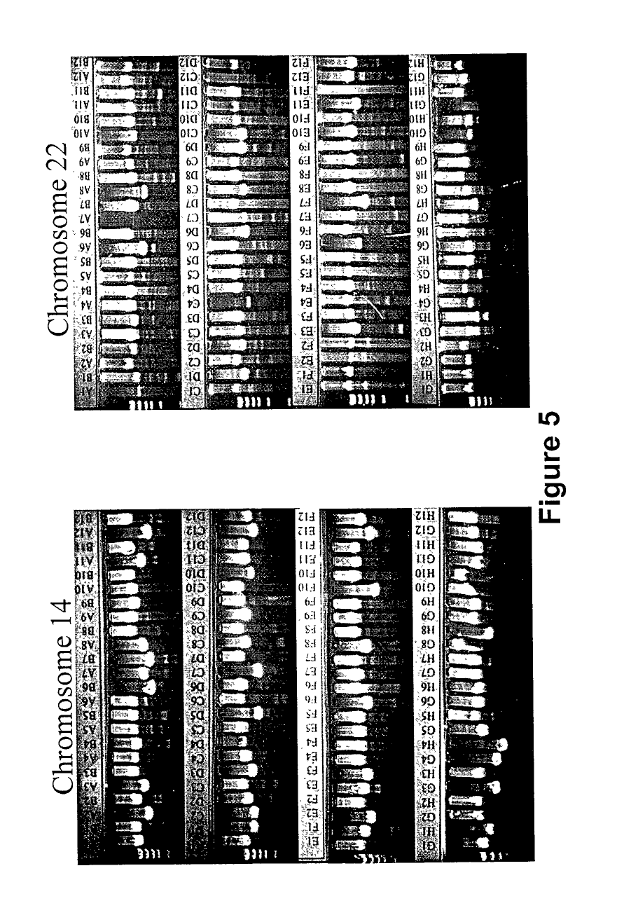

FIG. 5 shows two photographs of ethidium bromide stained agarose gels on which amplified, genomic DNAs from human chromosome 14 and chromosome 22 have been electrophoresed.

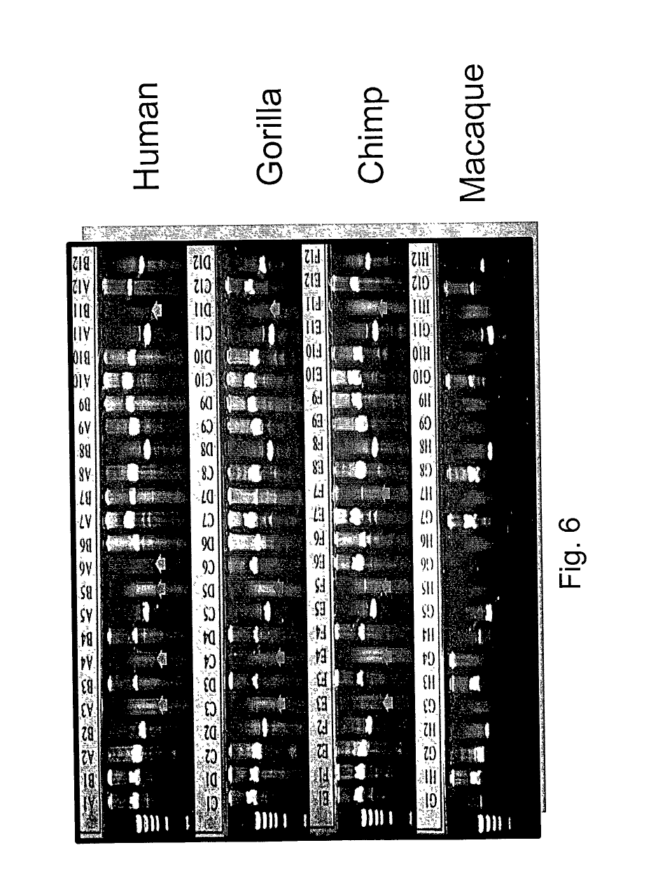

FIG. 6 shows photographs of ethidium bromide stained agarose gels on which amplified genomic DNA from human, gorilla, chimp, and macaque has been electrophoresed.

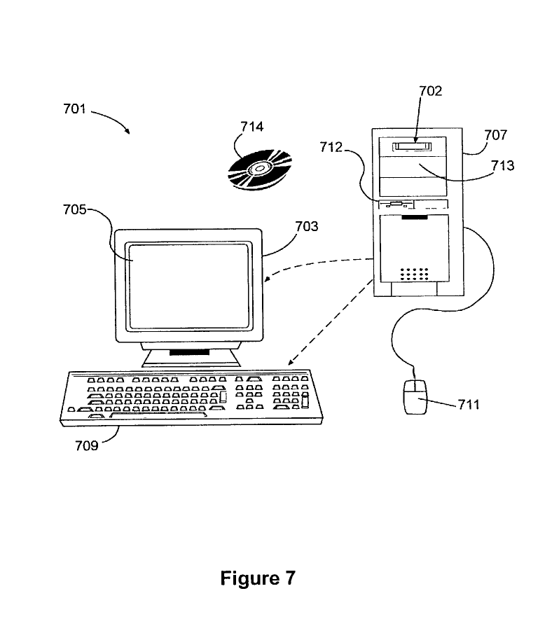

FIG. 7 shows a system that may be used for designing primer pairs.

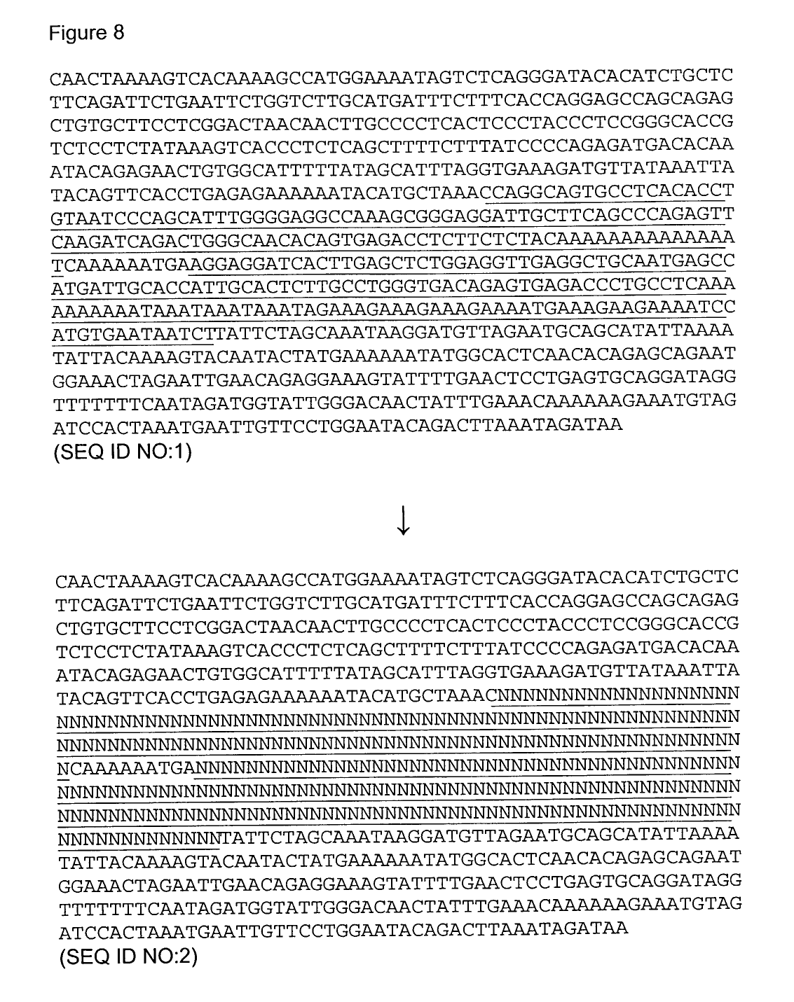

FIG. 8 shows an exemplary sequence before and after masking of repeat sequences (underlined).

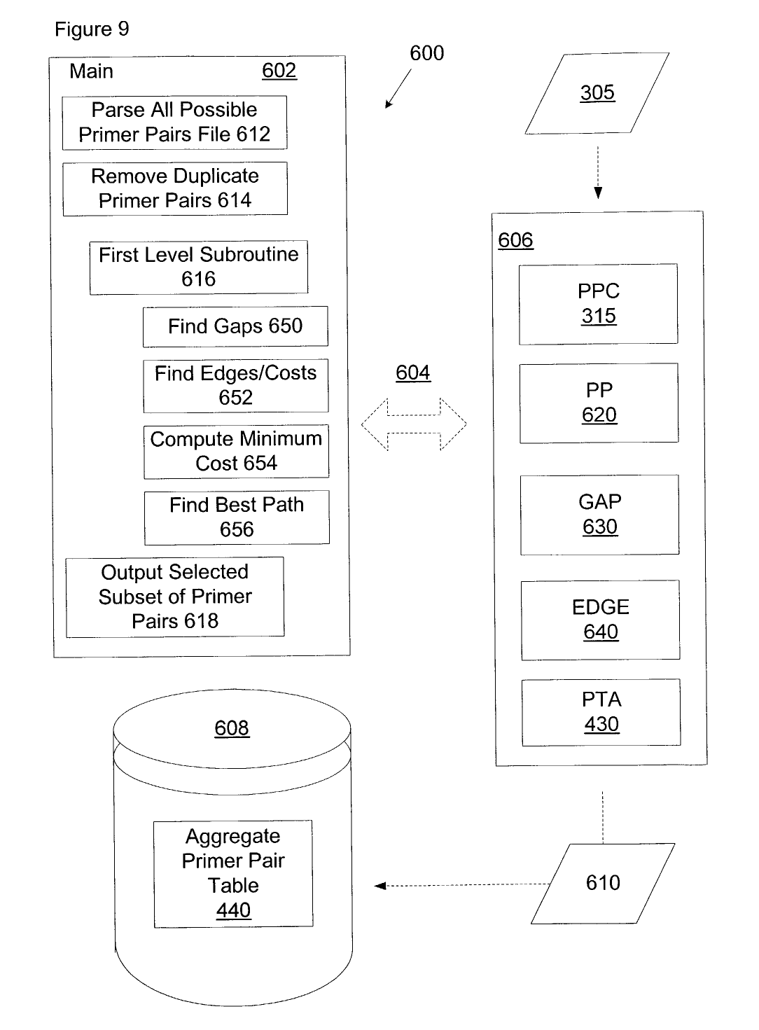

FIG. 9 shows a schematic block diagram illustrating the architecture of software implementing one embodiment of the invention.

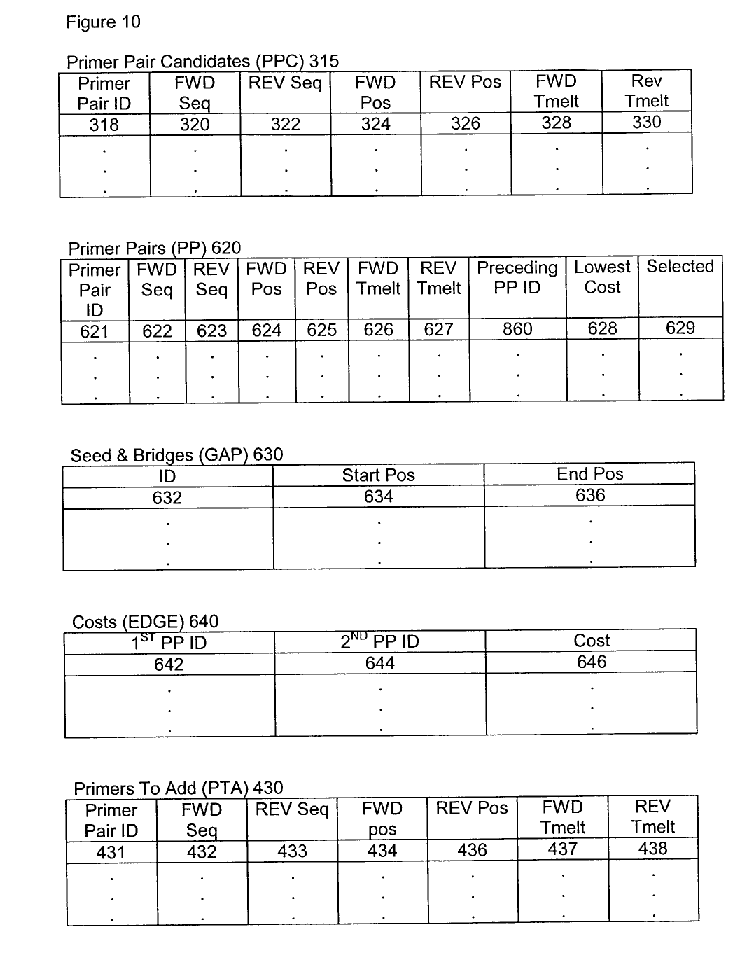

FIG. 10 shows a schematic diagram of a number of data structures used in the architecture shown in FIG. 9.

FIG. 11 shows a flow chart illustrating a detailed primer pair subset selection process according to one embodiment of the present invention.

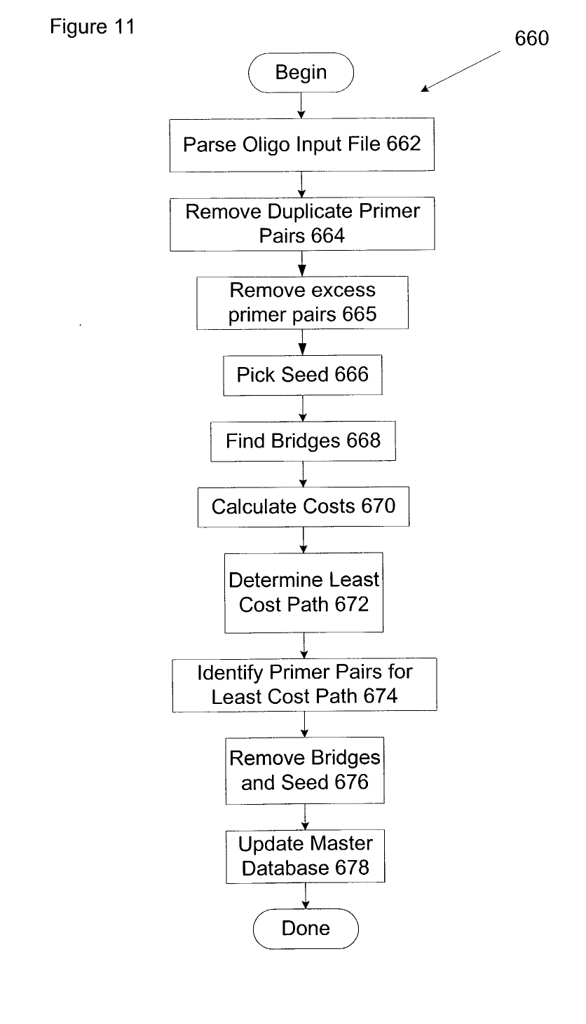

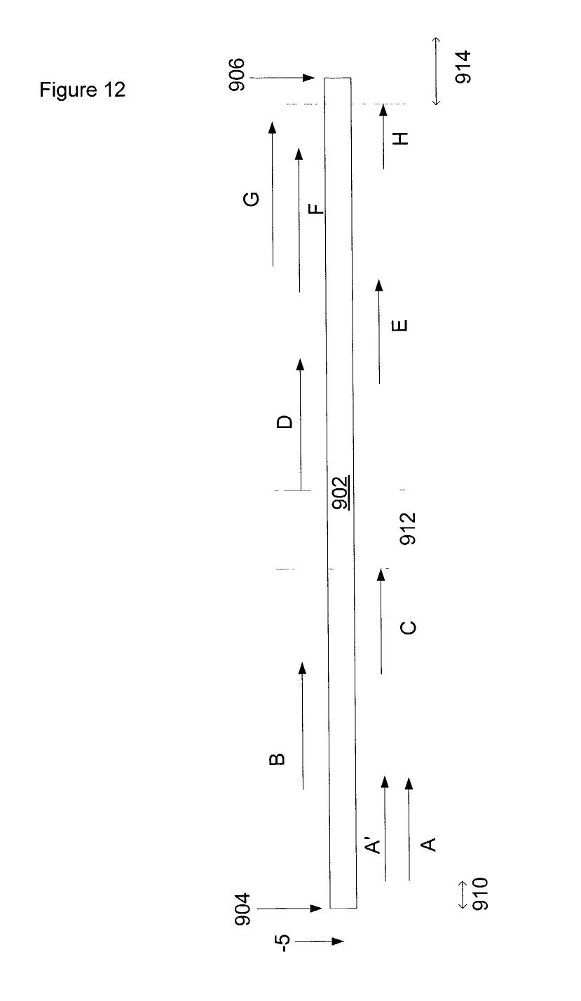

FIG. 12 shows a schematic illustration of a reference nucleic acid sequence and set of candidate primer pairs.

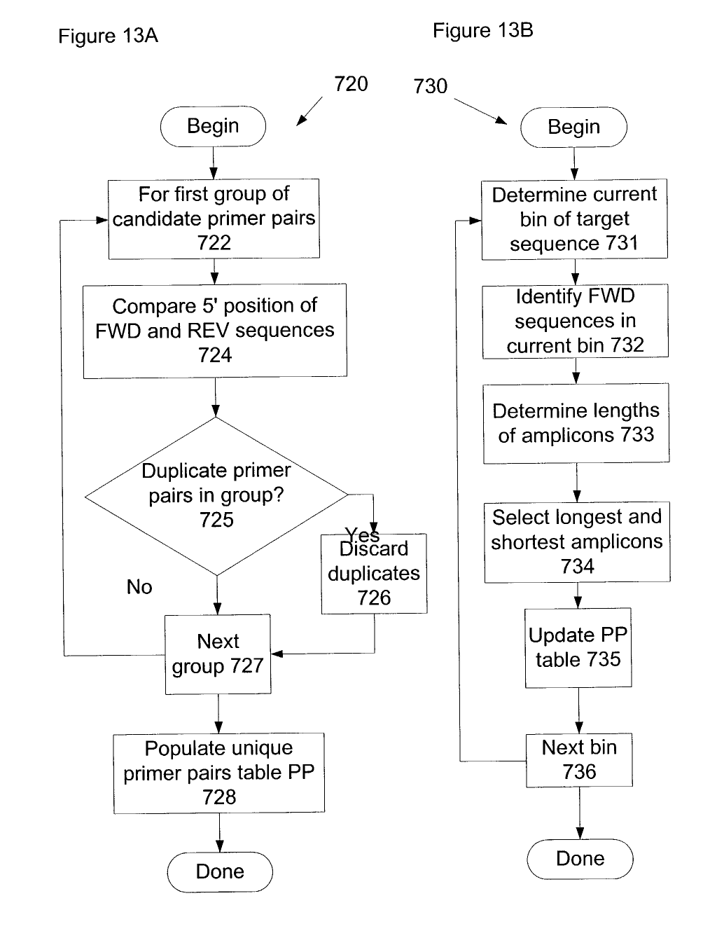

FIG. 13A shows a flow chart illustrating a duplicate primer pair reduction process in greater detail.

FIG. 13B shows a flow chart illustrating an optional excess primer pair reduction process in greater detail.

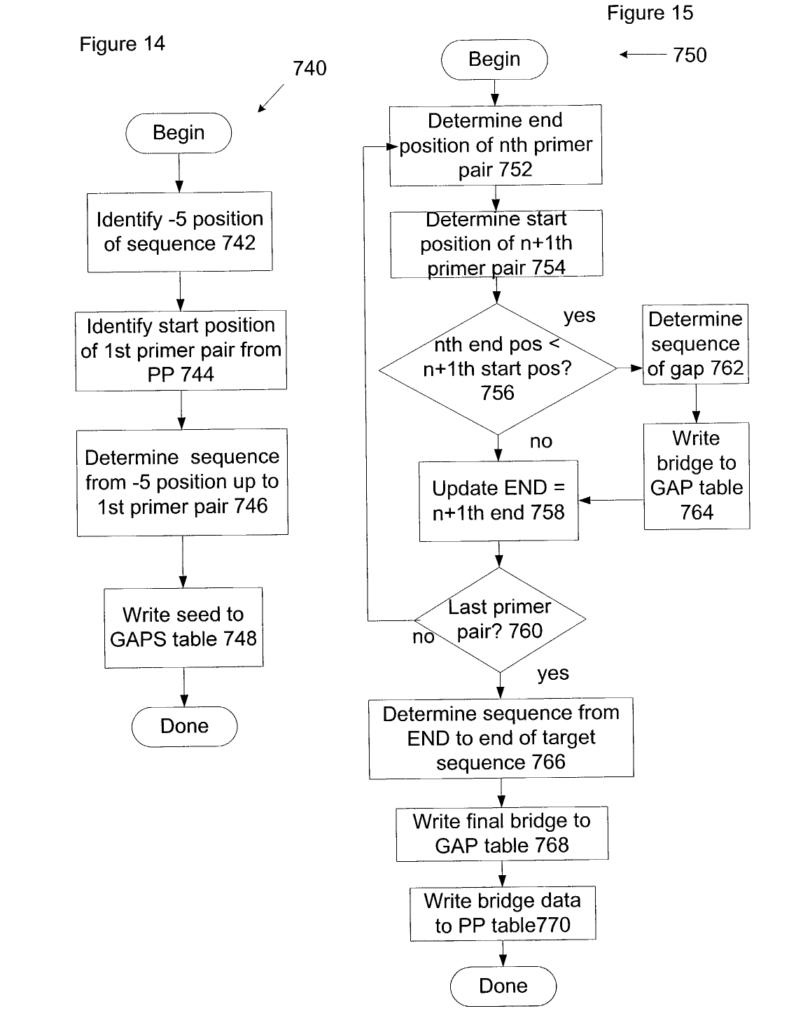

FIG. 14 shows a flow chart illustrating a seed picking process in greater detail.

FIG. 15 shows a flow chart illustrating a bridge finding process in greater detail.

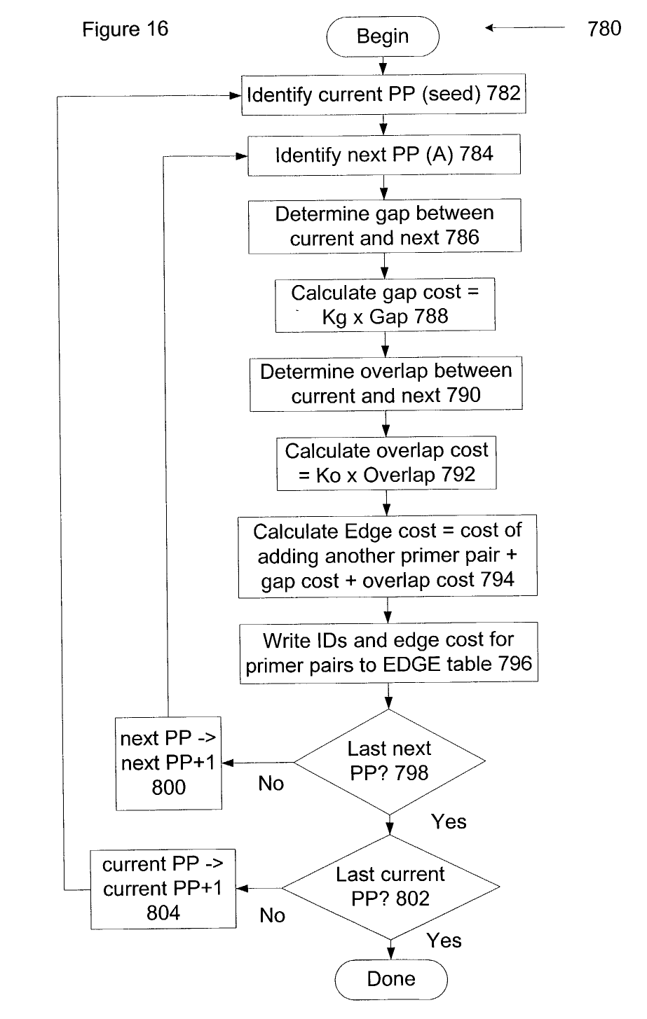

FIG. 16 shows a flow chart illustrating a cost calculating process in greater detail.

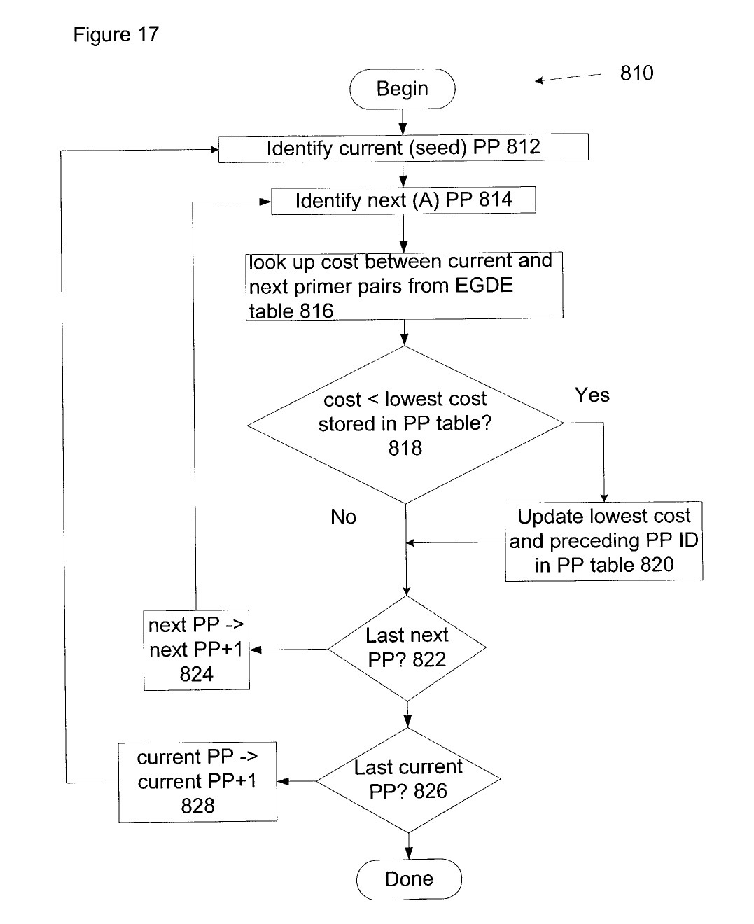

FIG. 17 shows a flow chart illustrating a primer pair lowest cost identifying process in greater detail.

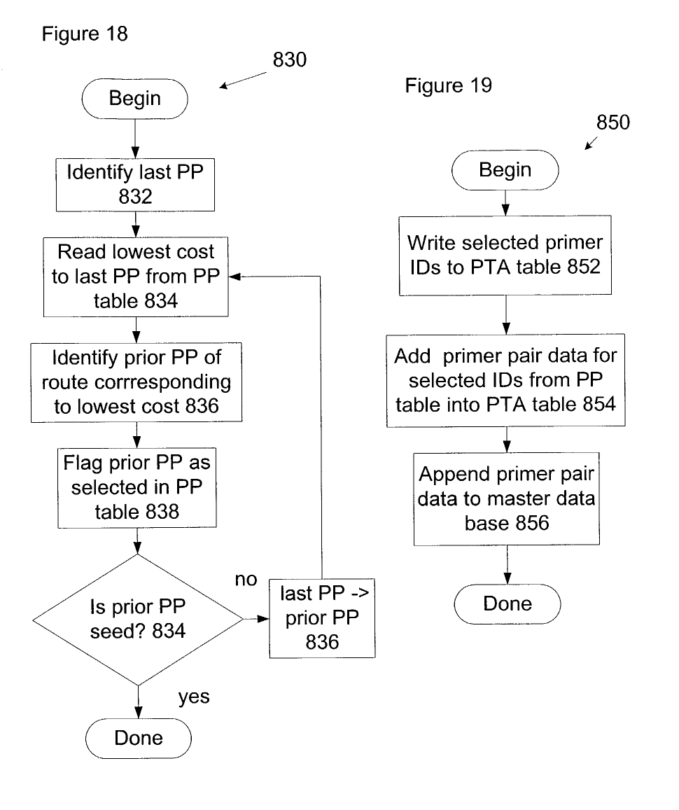

FIG. 18 shows a flow chart illustrating a primer pair subset selecting process in greater detail.

FIG. 19 shows a flow chart illustrating an output results process in greater detail.

### DETAILED DESCRIPTION OF EMBODIMENTS OF THE INVENTION

Reference now will be made in detail to various embodiments and particular applications of the invention. While the invention will be described in conjunction with the various embodiments and applications, it will be understood that such embodiments and applications are not intended to limit the invention. On the contrary, the invention is intended to cover alternatives, modifications and equivalents that may be included within the spirit and scope of the invention. In addition, throughout this disclosure various patents, patent applications, websites and publications are referenced. Unless otherwise indicated, each is incorporated by reference in its entirety for all purposes.

The term “a” or “an” as used herein in the specification may mean one or more. As used herein in the claim(s), when used in conjunction with the word “comprising”, the words “a” or “an” may mean one or more than one. As used herein “another” may mean at least a second or more.

Robust methods for designing primers and amplifying target sequences are described herein. In one specific embodiment of the present invention, amplification of between about 3 kilobases and about 15 kilobases or more in length has been achieved. The methods result in excellent fidelity of amplification and product yield for target sequences in general. In some applications of the present invention, the methods result in a greater than 95% success rate for amplification of mammalian genomic sequences genome-wide when a reference sequence and a target sequence are from the same species. However, in addition, the methods of the present invention can be used to amplify long target sequences genome-wide in species closely-related to the species from which a reference sequence was taken. For example, human sequence can be used to design primers that will produce long-range amplification products of non-human primates with a success rate of greater than 80%.

I. Primer Design

One aspect of the invention is methods for primer design. FIG. 1 is a flow chart generally illustrating the primer selection process. In step 100 of primer design, a sequence of interest (target sequence or reference sequence) is selected for amplification and downloaded into a sequence file (original sequence file). The sequence file and the software for performing the analysis herein may be stored on a computer system such as shown in FIG. 7.

In step 200, repeat sequences, such as Alu and LINE sequences in the reference sequence, are “masked” or removed from the primer selection analysis. In step 300, the non-repetitive, un-removed sequences that remain are analyzed according to at least two selection parameters and a set of all primer candidates that fit within the chosen parameters is established. Such selection parameters include, for example, melting temperature, likelihood of primer-dimer formation between the primers, primer length, and the like. Any of the primers generated by the third step may be used in the amplification reactions of the present invention.

In step 400, the set of primers generated by the third step is evaluated for coverage and overlap of the target sequence and a subset of primers is chosen so as to reduce the number of primers needed to amplify the target sequence.

A. Generation of a Primer Set

In the first step 100, a sequence of interest (target sequence) may be obtained, for example, from public databases such as the Human Genome Project Working Draft team at the University of California at Santa Cruz, NCBI, The Sanger Center, Whitehead Institute for Biomedical Research Center for Genome Research, Washington University Genome Sequencing Center, US DOE Joint Genome Institute, or Riken Gene Bank. Sequence generated de novo also may be used.

The second step 200 may be performed by hand or by a computer software program such as, for example, the program available from the University of Washington called “RepeatMasker”, a program that recognizes sequences that are repeated in the genome (A. F. A. Smit and P. Green,

www.genome.washington.edu/uwgc/analysistools/repeatmask,

incorporated herein by reference). Essentially, RepeatMasker screens genomic sequences for repeat regions in DNA, referencing a database of known repetitive elements called RepBase. RepBase Version 5 has been employed in the methods of the present invention, as have earlier versions of RepBase. The RepBase database can be licensed from the Genetic Information Research Institute (see www.girinst.org, incorporated herein by reference). Essentially, known repetitive sequences such as Single Interspersed Nuclear Elements (STNEs, such as alu and MIR sequences), Long Interspersed Nuclear Elements (LINEs such as LINE1 and LINE2 sequences), Long Terminal Repeats (LTRs such as MaLRs, Retrov and MER4 sequences), Transposons, MER1 and MER2 sequences are “masked” or removed by the RepeatMasker program by substituting each specific nucleotide of the repeated regions (A, T, G or C) with an “N” or “X”. In addition, xprimer (alces.med.umn.edu, Virtual Genome Center, incorporated herein by reference), a primer selection tool described below, can be used to identify simple, complex and internal repeats from a small database of repeats. Also, NCBI offers an Electronic PCR feature through its website (ncbi.nlm.nih.gov, incorporated herein by reference). The Electronic PCR program removes repetitive sequences from a non-repetitive marker set.

FIG. 8 shows an exemplary sequence with repeat regions shown (underlined), then removed or “masked” by inserting “Ns”. After the repeat regions are removed, primer pair candidates are selected from the unremoved sequence according to various parameters.

The third step 300 may be performed by hand or by a computer software program. For example, commercially available software such as Primer 3 (www-genome.wi.mit.edu/cgi-bin/primer/primer3, incorporated herein by reference), xprimer (alces.med.umn.edu, Virtual Genome Center, incorporated herein by reference), Oligo (Molecular Biology Insights, Inc., Cascade, Colo., incorporated herein by reference) or PrimerSelect (DNAStar, Inc., Madison, Wis., incorporated herein by reference) may be employed. Those with skill in the art may be familiar with other programs that are available for primer selection or can develop such a program. In one embodiment, a software program is used that allows one to dictate various primer parameters such as primer melting temperature, primer length, stringency of hybridization, existence of duplexes, specificity of hybridization, existence of a GC clamp, existence of hairpins, existence of sequence repeats, the dissociation minimum for a 3′ dimer, the dissociation minimum for the 3′ terminal stability range, the dissociation minimum for a minimum acceptable loop, percent maximum homology, percent consensus homology, the maximum number of acceptable sequence repeats, frequency threshold, or the maximum length of acceptable dimmers and the like. Also, in choosing primers for the third step, the length of a first primer of a primer pair may be fixed at a specific length, and the length of a second primer of the primer pair may be adjusted so that the melting temperature of the second primer pair is substantially the same as the melting temperature of the first primer.

Primer3 is a computer program that suggests PCR primers for a variety of applications, for example, to create STSs (sequence tagged sites) for radiation hybrid mapping, or to amplify sequences for SNP discovery. Primer3 also can select single primers for sequencing reactions and can design oligonucleotide hybridization probes. In selecting oligos for primers or hybridization probes, Primer3 can consider many factors, including oligo melting temperature, length, GC content, 3′ stability, estimated secondary structure, the likelihood of annealing to or amplifying undesirable sequences (for example interspersed repeats), the likelihood of primer-dimer formation between two copies of the same primer, and the accuracy of the source sequence. In the design of primer pairs, Primer3 can consider product size and melting temperature, the likelihood of primer-dimer formation between the two primers in the pair, the difference between primer melting temperatures, and primer location relative to particular regions of interest or regions to be avoided.

xprimer is another tool for selection of PCR primers. It is designed for selection of sets of primers along very large queries, where the primers must all fall within a relatively narrow melting temperature range. It is also useful in more traditional PCR applications. In xprimer, the actual primer sequences are printed to standard output with some statistical information. At the bottom of the display, a trace shows the log probability of the 3′ end of the sequence occurring in genomic DNA as determined using a preformed database.

PrimerSelect is a suite of tools for the design and analysis of oligonucleotides, including primers for PCR, sequencing, probe hybridization and transcription. Using DNA, RNA or back-translated proteins as templates, PrimerSelect details thermodynamic properties for annealing reactions. The software lists all possible primers, ranked in order of suitability. PrimerSelect includes a virtual lab where one can predict the effects the selected primers on reading frames, restriction sites and other features. Additionally, PrimerSelect allows for loading sequences directly from NCBI's databases, so that primers may be designed for published sequence.

Oligo is a multi-functional program that searches for and selects oligonucleotides from a sequence file for PCR sequencing, site-directed mutagenesis, and various hybridization applications. Oligo calculates hybridization temperature and secondary structure of oligonucleotides based on the nearest neighbor change in free energy values.

B. Selection of a Subset of Primer Pairs

The fourth step of primer design involves evaluating the set of primer pairs generated in steps one through three for coverage and overlap of the target sequence, and selecting a subset of primer pairs from the set of primer pairs. This fourth step may be performed by hand or by a computer software program. Typically the goal of the fourth step is to choose the primer pairs that allow one to amplify all or substantially all of the entire target sequence with reduced sequence amplification overlap and/or a minimal or substantially minimal number of primer pairs.

In preferred embodiments, the algorithm is used to select primers that will amplify more than 90% of the unremoved target sequence, preferably more than 95% percent of the unremoved target sequence, and preferably more than 99% percent. Preferably the amplified portions of the unremoved target sequence overlap by less than 5%, preferably less than 2% and preferably less than 1%. Preferably a minimum or near minimum number of primer pairs is used.

Algorithms known in the art may be applied for this purpose. For example, shortest path algorithms may be used (see, generally, Introduction to Algorithms, Cormen, Leiserson, and Rivest, MIT Press, 1994, pp. 514-578, incorporated herein by reference). In a shortest-paths problem, a weighted, directed graph G=(V,E), with weight function w: E→R mapping edges to real-valued weights is given. The weight of path p=(v₀, v₁, . . . vₖ) is the sum of the weights of its constituent edges:

$$w ( p ) = {\sum }_{i = 1}^{k} w ( {v}_{i - 1} , {v}_{i} ) .$$

The shortest-path weight from u to v is defined by δ(u,v) being equal to min w(p):u→v if there is a path from u to v, otherwise, δ(u,v) is equal to infinity. A shortest path from vertex u to vertex v is then defined as any path p with weight w(p)=δ(u,v). Edge weights can be interpreted as various metrics; for example, distance, time, cost, penalties, loss, or any other quantity that accumulates linearly along a path that one wishes to minimize. In the embodiment of the shortest path algorithm used in applications of this invention, each primer pair was considered a “vertex”. Each primer pair vertex has a relationship to each other primer pair vertex. This relationship is an “edge” defined for each pair of vertices, with a weight or “cost” for each edge. Cost is determined by parameters of choice, such as the extent of overlap of the vertices, the extent of gap between the vertices and a cost of adding another set of vertices to the final solution.

Single-source shortest-paths problems focus on a given graph G=(V,E), where a shortest path from a given source vertex sεV to every vertex vεV is determined. Additionally, variants of the single source algorithm may be applied. For example, one may apply a single-destination shortest-paths solution where a shortest path to a given destination vertex t from every vertex v is found. Reversing the direction of each edge in the graph reduces this problem to a single-source problem. Alternatively, one may apply a single-pair shortest-path problem where the shortest path from u to v for given vertices u and v is found. If the single-source problem with source vertex u is solved, the single-source shortest path problem is solved as well. Also, the all-pairs shortest-paths approach may be employed. In this case, a shortest path from u to v for every pair of vertices u and v is found—essentially, a single-source algorithm is run from each vertex.

One single-source shortest-path algorithm that may be employed in the methods of the present invention is Dijkstra's algorithm. Dijkstra's algorithm solves the single-source shortest-paths problem on a weighted, directed graph G=(V,E) for the case in which all edge weights are normegative. Dijkstra's algorithm maintains a set of vertices, S, whose final shortest-path weights from a source s have already been determined. That is, for all vertices v being elements of S, w[v]=δ(s,v). The algorithm repeatedly selects the vertex u as an element of V-S with the minimum shortest-path estimate, inserts u into S, and relaxes all edges radiating from u. In one implementation, a priority queue Q that contains all the vertices in V-S, keyed by their d values, is maintained. This implementation assumes that graph G is represented by adjacency lists.

Dijkstra (G, w, s)

1 INITIALIZE-SINGLE SOURCE (G,s)

2 S←Ø

3 Q←V[G]

4 while Q≠Ø

5 do u←EXTRACT-MIN (Q)

6 S←S U {u}

7 for each vertex vεAdj[u]

8 do RELAX (U, V, W)

Thus, G in this case is the graph of linear coverage of the target sequence, Q is the queue of all vertices to be evaluated and S is the set of vertices selected. Once one set of vertices (pair of primer pairs) is selected that covers a particular area of the target sequence, the other vertices that include these pairs can be discarded.

Other algorithms that may be used for selecting the subset of primers include a greedy algorithm (again, see, Introduction to Algorithms, Cormen, Leiserson, and Rivest, MIT Press, 1994, pp. 329-355). A greedy algorithm obtains an optimal solution to a problem by making a sequence of choices. For each decision point in the algorithm, the choice that seems best at the moment is chosen. This heuristic strategy does not always produce an optimal solution. Greedy algorithms differ from dynamic programming in that in dynamic programming, a choice is made at each step, but the choice may depend on the solutions to subproblems. In a greedy algorithm, whatever choice seems best at the moment is chosen and then subproblems arising after the choice is made are solved. Thus, the choice made by a greedy algorithm may depend on the choices made thus far, but cannot depend on any future choices or on the solutions to subproblems. In this case, the algorithm is “greedy: in selecting the “best” primer pair at a moment in time according to selected criteria, without regard to how this selection will affect which primer pairs are available for future selection.

One variation of greedy algorithms is Huffinan codes. A Huffinan greedy algorithm constructs an optimal prefix code and the algorithm builds a tree T corresponding to the optimal code in a bottom-up manner. It begins with a set C of leaves and performs a sequence of |C|-1 “merging” operations to create the final tree. For example, assuming C is a set of n characters and that each character cεC is an object with a defined frequency f[c], a priority queue Q, keyed on f is used to identify the two least-frequent objects to merge together. The result of the merger of two objects is a new object whose frequency is the sum of the frequencies of the two objects that were merged. For example:

1 n←|C|

2 Q←C

3 for i←1 to n−1

4 do z←ALLOCATE-NODE( )

5 x←left[z]←EXTRACT-MIN(Q)

6 y←right[z]←EXTRACT-MIN(Q)

7 f[z]←f[x]+f[y]

8 INSERT (Q,z)

9 return EXTRACT-MIN(Q)

Line 2 initializes the priority queue Q with the characters in C. The for loop in lines 3-8 repeatedly extracts the two nodes x and y of lowest frequency from the queue, and replaces them in the queue with a new node z representing their merger. The frequency of z is computed as the sum of the frequencies of x and y in line 7. The node z has x as its left child and y as its right child. After n−1 mergers, the one node left in the queue-the root of the code tree—is returned in line 9.

Thus, one aspect of the present invention provides a method for designing primer pairs for amplifying a target sequence, comprising the steps of choosing a reference sequence; removing selected repeat regions in the reference sequence to yield removed and unremoved reference sequences; selecting primer sequences from the unremoved reference sequences according to one or more parameters to yield a set of primers; evaluating the set of primers for extent of overlap and coverage of the reference sequence; and selecting a subset of primer pairs having reduced overlap from the set of primers. In one embodiment of this aspect of the invention, the removing step is performed by a computer program that references a database of known repeat sequences. In a specific embodiment of this aspect of the invention, the database is RepBase. Also in a specific embodiment of the present invention, the computer program that performs the removing step is RepeatMasker. Another embodiment of this aspect of the present invention provides that one of the one or more parameters from the first selecting step be, for example, parameters available for selection in commercially-available primer selection programs such as Oligo, xprimer, PrimerSelect, Primer 3 and the like. Such parameters include primer melting temperature, primer length, stringency, existence of duplexes, specificity, GC clamp, existence of hairpins, existence of sequence repeats, dissociation minimum for 3′ dimer, dissociation minimum 3′ terminal stability range, dissociation minimum for minimum acceptable loop, percent maximum homology, percent consensus homology, maximum number of acceptable sequence repeats, frequency threshold, or maximum length of acceptable dimers.

Also, in an embodiment of the present invention, the second selecting step selects a subset of primer pairs where this subset has a reduced number of primer pairs required to amplify the target sequence. Preferably, the subset is a substantially minimal number of primer pairs required to amplify the target sequence. In one embodiment, the second selecting step selects the subset of primer pairs according to additional parameters such as length of the overlap of the target sequence amplified by the primer pairs, existence of gaps of target sequence between primer pairs, and the necessity of adding another primer pair to the subset. In an embodiment of this aspect of the invention, the second selecting step is performed by a computer program. Such a program may apply a shortest-paths algorithm or greedy algorithm, and in one embodiment of the present invention, the computer program applies Dijkstra's single-source shortest paths algorithm (see FIGS. 2 and 3).

FIG. 2 shows one embodiment of the process in FIG. 1 in greater detail. At step 100, the target or reference sequence is downloaded from, for example, a public database, and stored in an original sequence file (105). At step 200, repeat sequences in the target sequence are removed from the primer selection process by, for example, a computer program such as RepeatMasker. A file of the unremoved sequence (205) is stored on a server or similar memory device. At step 300, primer pair candidates are selected in accordance with established, selected parameters, and these primer pair candidates are stored in a file (305) on a server or similar memory device. Preferably, all possible primer pairs that fall within the established parameters are stored in file 305. At step 310, the file of all possible primer pairs is parsed, loaded and a candidate primer pair table (315) is generated. At step 400, a subset of primer pairs is selected by applying, for example, a greedy algorithm. The subset of primer pairs is stored in file 430, a “primers to add” table, on a server or similar memory device. The primers to add table is then appended to a master database in step 435, adding this subset of primer pairs to an aggregate primer pair table 440.

FIG. 3 shows greater detail of one embodiment of step 400, selecting a subset of primer pairs from the table of all primer pairs generated at step 300. Step 405 evaluates the table of all primer pairs generated at step 300, finding stretches of the target sequence where there are no primer pairs useful for amplification. Step 410 then adds fake primer pairs to cover these stretches so as to remove these gaps between primer pairs from the solution reached when applying the single-source shortest-path algorithm in steps 415, 420 and 425. Step 415 determines the cost of each “edge” according to pre-selected criteria for cost, step 420 finds the lowest cost for each set of primer pairs and step 425 finds the best path for amplifying the target sequence. The subset of primers generated by steps 405, 410, 415, 420, and 425 is then stored in a file 430 on a server or similar memory device.

II. Computer System

One embodiment of the present invention provides a computer program for designing primer pairs for amplifying a target nucleic acid sequence. The computer program comprises computer code that receives input of a reference sequence; computer code that removes selected repeat regions in the reference sequence; computer code that selects primer sequences from the unremoved reference sequence; computer code that evaluates the set of primers for extent of coverage and overlap of the reference sequence; and computer code that selects a subset of primer pairs having reduced overlap from the set of primers. Preferably, the computer code that selects primer sequences from the unremoved reference sequence selects sequences according to two or more parameters including primer length and primer melting temperature to yield a set of primers.

Another embodiment of the present invention provides a system that designs primer pairs for amplifying a target nucleic acid sequence. This system comprises a processor; and a computer readable medium coupled to the processor for storing a computer program. The computer program comprises computer code that receives input of a reference sequence; computer code that removes selected repeat regions in the reference sequence; computer code that selects primer sequences from the unremoved reference sequence; computer code that evaluates the set of primers for extent of coverage and overlap of the reference sequence; and computer code that selects a subset of primer pairs having reduced overlap from the set of primers. Preferably, the computer code that selects primer sequences from the unremoved reference sequence selects sequences according to two or more parameters including primer length and primer melting temperature to yield a set of primers.

For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems: A Practical Guide for Systems Professionals by Paul E. Renaud, 2nd edition (June 1996), John Wiley & Sons; ISBN: 0471133337, both are incorporated herein by reference in their entireties for all purposes.

Appendix 1 attached hereto provides an exemplary computer code in Visual Basic (Visual Basic is a trade mark of Microsoft Corporation and is registered in some countries). This code covers loading the candidate primer pairs (315), through adding the subset of selected primers to the primers-to-add table (step 430) (see FIGS. 1 and 2). FIG. 7 illustrates an example of a computer system that may be used to execute the software of an embodiment of the invention. FIG. 7 shows a computer system 701 that includes a display 703, screen 705, cabinet 707, keyboard 709, and mouse 711. Mouse 711 may have one or more buttons for interacting with a graphic user interface. Cabinet 707 houses a floppy drive 712, CD-ROM or DVD-ROM drive 702, central processing unit, system memory and a hard drive 713 which may be utilized to store and retrieve software programs incorporating computer code that implements the invention, data for use with the invention and the like. Although a CD 714 is shown as an exemplary computer readable medium, other computer readable storage media including floppy disk, tape, flash memory, system memory, and hard drive may be utilized. Additionally, a data signal embodied in a carrier wave (e.g., in a network including the Internet) may be the computer readable storage medium.

III. Amplification Reaction

In another aspect of the present invention, methods for long range nucleic acid amplification are provided, including cycling temperatures, cycling times, reagents and reagent concentrations. The methods allow for consistent long range amplification of sequences genome-wide. In some embodiments of the present invention, amplification of between about 3 kilobases and about 15 kilobases or more in length has been achieved. In some applications of the present invention, the methods result in a greater than 95% success rate for long range amplification of mammalian genomic sequences genome-wide when the reference sequence and the target sequence are from the same species. However, in addition, the methods of the present invention can be used to amplify long target sequences genome-wide in species closely-related to the species from which a reference sequence was taken. Various aspects of the present invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

FIG. 4 illustrates the basic steps of an amplification reaction. In step 500 of the amplification method, reagents, target and the selected primers are combined to form a reaction mixture. In step 505, the reaction mixture is heated to a temperature sufficient to denature the target nucleic acid, then cooled in step 510 to a temperature sufficient to allow annealing of the primers to the target and extension of the annealed primers. The heating step 505 and cooling step 510 then are repeated so as to amplify the target nucleic acid.

Also in certain embodiments of the present invention, an initial heating step may be added before the heating (505)/cooling (510) cycling where the reaction cocktail is heated at about 90° C. to about 96° C. for 1.0 to 10.0 minutes. In a preferred embodiment, this initial heating step is at about 95° C. for about 3.0 minutes. In an alternative embodiment of the present invention, the cooling time for cooling step 510 may be increased for each successive heating/cooling cycle. In one such embodiment, the cooling time is increased by about 1 to about 30 seconds in each successive cycle, and in a preferred embodiment, the cooling time is increased by about 20 seconds in each successive cycle.

In yet another embodiment of the present invention, an additional cooling step is performed after the heating (505)/cooling (510) cycle and before a final 4.0° C. cooling hold step, wherein the additional cooling step annealing/extension temperature is about 58° C. to about 65° C. and is performed for about 5 minutes to about 45 minutes. In a preferred embodiment the additional cooling step annealing/extension temperature is about 62° C. and performed for about 60 minutes.

In a specific aspect of the invention, the primers have a length of about 28 nucleotides to about 36 nucleotides and a melting temperature of about 72.0° C. to about 88.0° C. In this aspect, Tm was measured at a monovalent ion concentration of 1000 mM, a free Mg⁺⁺ concentration of 0.0 mM, a total Na⁺ equivalent of 1000 mM, a nucleic acid concentration of 100 pM and where the temperature for ΔG calculations was 25° C.

In one embodiment of the present invention, the reaction cocktail resulting from step 500 comprises deoxynucleotide triphosphates such as dATP, dTTP, dCTP, dUTP and dGTP or mimetics thereof, target DNA, a divalent cation, DNA polymerase enzyme, a broad spectrum solvent, a zwitterionic buffer and at least one primer pair designed by the primer selection methods described above. The heating step 505 is conducted at a denaturing temperature of about 90° C. to about 96° C., preferably of about 92° C. to about 95° C., and more preferably of about 94° C. The denaturing temperature of the heating step 505 is maintained for about 1 to about 30 seconds, preferably for about 1.5 to about 5 seconds, and more preferably for about 2 seconds. The cooling step 510 is conducted at an annealing/extension temperature of about 50° C. to about 68° C., preferably of about 58° C. to about 65° C., and more preferably of about 64° C. The annealing/extension temperature is maintained for about 1 minute to about 28 minutes, and preferably for about 12 minutes. The heating and cooling steps are repeated at least about 10 times and preferably about 25 to 45 times, or more preferably about 30 to 40 times. A final cooling of the reaction cocktail to 4° C. is performed after the final cooling step 510.

In an embodiment of the present invention, the reaction cocktail comprises about 50 μM to about 400 μM of each primer in the primer pair, preferably about 100 nM to about 240 nM of each primer in the primer pair, and more preferably about 192 nM of each primer in the primer pair. In addition, the reaction cocktail comprises about 200 μM to about 500 μM each dNTP, preferably about 300 μM to about 400 μM each dNTP, and more preferably about 385 μM each dNTP. The reaction cocktail also comprises about 0.02 ng/μl to about 2.5 ng/μl template (target) DNA, preferably about 0.05 ng/μl to about 1.5 ng/μl template (target) DNA, and more preferably about 1.2 ng/μl template (target) DNA. The reaction cocktail may also comprise 0.0% to about 7.0% broad spectrum solvent, preferably 1.5% to about 4.5% broad spectrum solvent, and more preferably about 3.7% broad spectrum solvent. In preferred embodiments, the broad spectrum solvent is DMSO.

Further, the reaction cocktail comprises 0.0 M to about 0.75 M betaine, preferably about 0.2 M to about 0.6 M betaine, and more preferably about 0.24 M betaine, and about 7 mM to about 35 mM NH₄SO₄, preferably about 10 mM to about 20 mM NH₄SO₄, and more preferably about 13 mM NH₄SO₄. The reaction cocktail also includes about 25 mM Tris to about 125 mM Tris, preferably about 40 mM Tris to about 80 mM Tris, and more preferably about 48 mM Tris, and about 100 μM to about 500 μM MgCl₂, preferably about 250 μM to about 400 μM MgCl₂, and more preferably about 385 μM MgCl₂.

The reaction cocktail also comprises a polymerase. In certain embodiments, the reaction cocktail comprises about 0.01 units/μl to about 0.2 units/μl polymerase, preferably about 0.025 units/μl to about 0.07 units/μl polymerase, and more preferably about 0.05 units/μl polymerase. In addition, the reaction cocktail may comprise about 0 mM to about 50 mM zwitterionic buffer, preferably about 10 mM to about 30 mM zwitterionic buffer, and more preferably about 25 mM zwitterionic buffer. In some embodiments, the zwitterionic buffer is Tricine.

Also in some embodiments, about 0.005 μg/μl to about 0.10 μg/μl taq antibody may be added to the reaction cocktail. Preferably, about 0.01 μg/μl to about 0.05 μg/μl taq antibody is added to the reaction cocktail, and more preferably about 0.017 μg/μl taq antibody is added to the reaction cocktail.

IV. Applicability to Diverse Sequences

PCR has been applied widely in molecular biology; however, despite such wide-spread use, amplifying varying long stretches of DNA is difficult. Many protocols for long range PCR exist; however, reaction conditions are usually optimized for amplifying specific target regions of interest. Similar amplification success is not achieved when these “optimized” reaction conditions are used on different target regions. In the present invention, however, amplification of between about 3 kilobases and about 15 kilobases or more in length has been achieved on varied genomic sequences genome-wide. The methods result in excellent fidelity of amplification and product yield for mammalian target sequences in general. In some applications of the present invention, the methods result in a greater than 95% success rate for amplification of mammalian genomic sequences when the reference sequence and the target sequence are from the same species. However, in addition, the methods of the present invention can be used to amplify long target sequences genome-wide in species closely-related to the species from which a reference sequence was taken. For example, human sequence can be used to design primers that will produce long-range amplification products of non-human primates with a success rate of greater than 80%.

FIG. 4 shows the results obtained with the methods of the present invention for human chromosome 14 sequence used as a reference sequence for primer design and human target DNA and human chromosome 22 sequence used as a reference sequence for primer design and human target DNA. FIG. 5 shows the results obtained with the methods of the present invention with human DNA used as a reference sequence for primer design and human, gorilla, chimpanzee, and macaque genomic DNA used as target sequences.

### V. EXAMPLES

The examples below illustrate specific implementations of the inventions described herein.

A. Preparation and Scoring of Somatic Cell Hybrids

Standard procedures in somatic cell genetics were used to separate human DNA strands (chromosomes) from a diploid state to a haploid state. Diploid human lymphoblast cell lines from a human diversity panel lymphoblast line (available from Coriell Cell Repositories, Camden, N.J.) were fused to a diploid hamster fibroblast cell line containing a mutation in the thymidine kinase gene. In a sub-population of the resulting fused cells, human chromosomes were introduced into the hamster calls. Selection for the human DNA-containing hamster cells (fusion cells) was achieved by utilizing HAT medium. Only hamster cells that had a stably incorporated human DNA strand grow in cell culture medium containing HAT.

Hamster cell line A23 cells were pipetted into a centrifuge tube containing 10 ml DMEM in which 10% FBS (fetal bovine serum)+1× Pen/Strep (penicillin/streptomycin)+10% glutamine were added, centrifuged at 1500 rpm for 5 minutes, resuspended in 5 ml of RPMI and pipetted into a tissue culture flask containing 15 ml RPMI medium. The lymphoblast cells were grown at 37° C. to confluence. At the same time, human lymphoblast cells were pipetted into a centrifuge tube containing 10 ml RPMI in which 15% FBCS+1× Pen/Strep+10% glutamine were added, centrifuged at 1500 rpm for 5 minutes, resuspended in 5 ml of RPMI and pipetted into a tissue culture flask containing 15 ml RPMI. The lymphoblast cells were grown at 37° C. to confluence.

To prepare the A23 hamster cells, the media was aspirated and the cells were rinsed with 10 ml PBS (phosphate-buffered saline). The cells were then trypsinized with 2 ml of trypsin and divided into 3-5 plates of fresh media (DMEM without HAT) and incubated at 37° C. The lymphoblast cells were prepared by transferring the culture into a centrifuge tube and centrifuging at 1500 rpm for 5 minutes, resuspending the cells in 5 ml RPMI and pipetting 1 to 3 ml of cells into 2 flasks containing 20 ml RPMI.

To achieve cell fusion, approximately 8-10×10⁶ lymphoblast cells were centrifuged at 1500 rpm for 5 min. The cell pellet was then rinsed with DMEM by resuspending the cells and centrifuging them again. The lymphoblast cells were then resuspended in 5 ml DMEM. The recipient A23 hamster cells had been grown to confluence and split 3-4 days before the fusion and were, at this point, 50-80% confluent. The old media was removed and the cells were rinsed 3 times with DMEM and finally suspended in 5 ml DMEM. The lymphoblast cells were slowly pipetted over the recipient A23 cells and the combined culture was swirled slowly before incubating at 37° C. for 1 hour. After incubation, the media was gently aspirated from the A23 cells, and 2 ml room temperature PEG 1500 was added by touching the edge of the plate with a pipette and slowly adding PEG to the plate while rotating the plate with the other hand. It took approximately 1.5 minutes to add all of the PEG in one full rotation of the plate. Next, 8 ml DMEM was added down the edge of the plate while rotating the plate slowly. The PEG/DMEM mixture was aspirated gently from the cells and then 10 ml DMEM was used to rinse the cells. This DMEM was removed and 10 ml fresh DMEM was added and the cells were incubated for 30 min. at 37° C. Again the DMEM was aspirated from the cells and 10 ml DMEM in which 10% FBS and 1× Pen/Strep were added, was added to the cells, which were then allowed to incubate overnight.

After incubation, the media was aspirated and the cells were rinsed with PBS. The cells were then trypsinized and divided among 20 plates containing selection media (DMEM in which 10% FBCS+1× Pen/Strep+1×HAT were added) so that each plate received approximately 100,000 to 150,000 cells. The media was changed on the third day following plating. Colonies were picked and placed into 24-well plates upon becoming visible to the naked eye (day 9-14). If a picked colony was confluent within 5 days, it was deemed healthy and the cells were trypsinized and moved to a 6-well plate.

DNA and stock hybrid cell cultures were prepared from the cells from the 6-well plate cultures. The cells were trypsinized and divided between a 100 mm plate containing 10 ml selection media and an eppendorf tube. The cells in the tube were pelleted, resuspended 200 μl PBX and DNA was isolated using a Qiagen DNA mini kit at a concentration of <5 million cells per spin column. The 100 mm plate was grown to confluence, and the cells were either continued in culture or frozen.

Scoring for the presence, absence and diploid/haploid state of each hybrid was performed using the Affymetrix, Inc. HuSNP GENECHIP® (Affymetrix, Inc. of Santa Clara, Calif., GENECHIP® HuSNP Mapping Assay, reagent kit and user manual, Affymetrix Part No. 900194), which can score 1,494 markers in a single chip hybridization. As a control, the human diploid lymphoblast cell line was screened using the HuSNP chip hybridization assay, and any SNPs which were heterozygous in the parent lymphoblast diploid cell line were scored for haploidy in each fusion cell line. By comparing the markers that were present as “AB” heterozygous in the parent diploid cell line to the same markers present as “A” or “B” (hemizygous) in the hybrids, the human DNA strands which were in the haploid state in each hybrid line was determined.

B. Primer Selection

Human genomic sequence was used as a reference sequence for primer selection in this example of the present invention, and human genomic DNA derived from somatic cell hybrids was used as target DNA. In addition, in an alternative application of the present invention, human genomic sequence was used as reference sequence for primer selection and genomic DNA from gorilla and chimpanzee was used as target DNA.

FIG. 2 is a flow chart showing a detailed primer selection process according to one embodiment of the present invention. The first step 100 of primer selection required selecting a sequence of interest (target sequence or reference sequence) and creating an original sequence file (105) containing this selected sequence. Next, repeat regions in the target sequence were removed (200), and a removed file was created containing the unremoved sequence (205). In the third step, the sequences in the removed file were run through a primer pair selection program (300) using primer parameters chosen by the user, and the set of all possible primers meeting the primer parameters was generated and stored in an oligo output file (305). The information from the oligo output file was then used to create a candidate primer pair table (315). In step four of the selection process (400), an optimal subset of primer pairs was selected from the set of all possible primer pairs in the primer pair table. The output from the selection of the optimal subset of primer pairs was stored in the primers to add table (430), which was then appended to the master database (435) and stored in an aggregate primer pair table (440).

First, human sequence to be used as the reference sequence for primer design was acquired from the Human Genome Project Working Draft team from the University of California at Santa Cruz where sequence assembly was performed using sequences obtained from the High Throughput Genomic Sequence (HTGS) database. The HTGS database is a public database with sequences contributed by, inter alia, the Human Genome Project Working Draft team. The UTSC assembly is available at the UCSC site [http://genome.cse.ucsc.edu/], and a detailed description of the data format can be found at [http://genome.cse.ucsc.edu/goldenPath/datorg.html]. Sequence was also acquired from NCBI.

In the second step, acquired reference sequence was processed by a software program called “RepeatMasker”, available for licensing from the University of Washington (see:A. F. A. Smit and P. Green,

[www.genome.washington.edu/uwgc/analysistools/repeatmask.htm]).

RepeatMasker screens genomic sequences for repeat regions in DNA, referencing a database of known repetitive elements called RepBase. RepBase Version 5 was employed in the methods of the present invention, as were earlier versions of RepBase. The RepBase database was licensed from the Genetic Information Research Institute (see www.girinst.org). Known repetitive sequences such as Single Interspersed Nuclear Elements (SINEs, such as alu and MIR sequences), Long Interspersed Nuclear Elements (LINEs such as LINE1 and LINE2 sequences), Long Terminal Repeats (LTRs such as MaLRs, Retrov and MER4 sequences), Transposons, MER1 and MER2 sequences were “masked” or removed by the RepeatMasker program by substituting each specific nucleotide of the repeated regions (A, T, G or C) with an “N” or “X”. Local nucleotide duplications were not masked. In one application of the present invention, the default settings of RepeatMasker were used, and the human.ref library (human repetitive elements) and simple.ref library were concatenated and combined to SnRNAs from the pseudo.ref library to create a “custom” library. Those skilled in the art will appreciate that any computer program, algorithm or selection process, including manual selection, which identifies and eliminates from primer selection repetitive sequences from the reference sequence may be used as an alternative to RepeatMasker.

Once the reference sequence was masked and repetitive regions removed, a third step was performed where the masked sequence output was then entered into the commercially-available primer design program, Oligo 6.52 using the following search parameters:

Search For: Primers and Probes

±Strand Search

Select:

Complex Substrate

Compatible Pairs

Duplex-free Oligonucleotides

Highly Specific Oligos [3′-end stability]

Oligonucleotide with GC Clamp

Eliminate False Priming Oligonucleotides

Oligonucleotides within Selected Stability Limits

Hairpin-free Oligonucleotides

Eliminate Homooligomers/Sequence Repeats

Eliminate Frequent Oligos

Search Mode:Mark

PCR Product Length: 3000 to 15000

General Settings:

High Search Stringency

No Auto Change

Adjust Length to Match Tm's

Parameters:

Oligonucleotide Length: 32 nt

Acceptable 3′-Dimer ΔG: −3.5 kcal/mol

Maximum Length of Acceptable Dimers: 4 Base Pairs

3′-terminal Nucleotides Checked for Dimers: 23

3′-terminal Stability Range: −5.5 to −9.8 kcal/mol

GC Clamp Stability: −10.0 kcal/mol

Minimum Acceptable Loop ΔG: 0.0 kcal/mol

Oligo Tm Range [58.1 to 108.1]: 72.0 to 88.0° C.

Max Acceptable False Priming Efficiency: 170 Points

Min Consensus Priming Efficiency: 340 Points

Max Acceptable Homology: 50%

Min Consensus Homology: 95%

Max Number of Acceptable Sequence Repeats: 3

Max Degeneracy: 1

Frequency Threshold: 1000

Non-Search Parameters:

Monovalent Ion Concentration: 1000 mM

Free Mg⁺⁺ Concentration: 0.0 mM

Total Na⁺⁺ Equivalent: 1000 mM

Nucleic Acid Concentration: 100 pM

Temperature for ΔG Calculations: 25° C.

All possible primer pairs generated within the established parameters were saved to a file. Any of the generated primer pairs may be used in the amplification reactions of the present invention; however, typically primer pairs will be chosen that cover as much of the reference sequence as possible with reduced overlap.

In the present embodiment, the primer pair set output obtained from Oligo 6.52 was, in the fourth step of primer selection, subjected to Dijkstra's algorithm (again, see Introduction to Algorithms, Cormen, Rivest and Leiserson (1990); ISBN 0262031418)). The goal of this step being to find a best subset of primer pairs to amplify the target sequence out of all possible sets of primer pairs generated by Oligo 6.52. Dijkstra's algorithm solves the single-source shortest path problem on a weighted, directed graph. In the embodiment of this algorithm used in applications of this invention, each primer pair was considered a “vertex” with an “edge” defined for each pair of vertices. An associated “cost” was assigned to each edge where the cost reflected the amount of: 1) the overlap of vertices (cost=the length of the overlap); 2) the gap between two primer pairs (cost=10×the length of the gap); and 3) a fixed value for having to add another vertex to the set (which increased the number of primers that must be used) (cost for additional primer pair=4000). In one application of the present invention, the path with the lowest cost was selected, where total cost equals the sum of the costs of edges in the path. For example, assume three exemplary primer pairs:

| | 5′ position | 5′ position |
|---|---|---|
| | of the forward primer | of the reverse primer |
| Primer 1: | 1000 | 2000 |
| Primer 2: | 1800 | 3000 |
| Primer 3: | 2100 | 4000 |

The “edges” are defined as being between Primer 1 and Primer 2, Primer 1 and Primer 3, and Primer 2 and Primer 3. The cost associated with the edge Primer1/Primer2 is 200+0 (100)+4000=4200 (reflecting the 200 base overlap between the amplicons). The cost associated with edge Primer1/Primer3 is 0+10 (100)+4000=5000 (reflecting the 100 base pair gap between Primer 1 and Primer 3). The cost associated with edge Primer2/Primer 3 would be 900+0(100)+4000=4900 (reflecting the 900 base overlap between the amplicons).

In one embodiment of the present invention, the computer code for evaluating the primer set for extent of coverage and overlap of the reference sequence and selecting the subset of primer pairs was comprised of a main module, a first level subroutine, and several second level subroutines. This code is reproduced below.

FIG. 9 is a schematic block diagram illustrating the architecture 600 of an embodiment of the software implementing a method for selecting primer pairs. Computer code 602 is executed by a general purposed digital computer 701 to carry out the steps of the method. Computer code 602 reads and writes 604 data items held in a number of tables 606 stored in a random access storage device, such as the memory or hard drive 713 of computer 701. The computer code can also output results 610 to the aggregate primer pair table 440 in the master database 608.

In a preferred embodiment the tables 606 are in an Access database (Access is a trade mark of Microsoft Corporation) and the computer code 602 is written in VBA, a version of Visual Basic particularly suitable for use with Access.

The main module, Main, includes computer code 612 to parse and load the file of all possible primer pairs 305 for the masked reference sequence from the third step 300. Computer code 614 is provided to reduce the number of candidate primer pairs if there are a significant number of very similar primer pairs, so as to improve the speed of processing. The main program includes code to run a first level subroutine 616, and then 618 take the information output 610 from the first level subroutine and append this information to a local repository of information, which ultimately is copied to the aggregate primer pair table 440.

The first level subroutine, Select Optimal Primers 616, directs several second level subroutines, which essentially applied Dijkstra's algorithm to select a subset of primer pairs from the set of all possible primer pairs (see FIG. 3). Select Optimal Primers retrieves the information from the primer pair table 620 (parsed Oligo Results Files), and includes code 650 to find gaps in the primer pair amplification coverage of the reference sequence (Find Gaps 405). Fake primer pairs or bridges are added to the data to cover the gaps so as not to penalize the solution for the subset selection for an unavoidable gap (Add Fake Primer Pairs for Gaps 410). Computer code 652, determines a cost for each edge (Find Edges 415), code 654 computes the lowest cost for every possible set of primer pairs (Compute Minimum Costs 420), and code 656 to find the best subset of primer pairs (Find Best Path 425). The results are output by code 618 which adds this subset of primer pairs to a local repository 430 which are then added to the final aggregate repository of primer pairs 440.

FIG. 10 illustrates the structure of the tables 606 used to hold the various data items processed by the program 602. A primer pair candidates (PPC) table 315 holds data item relating to the candidate primer pairs identified by the Oligo primer picking program for amplifying the target sequence. In this embodiment of the invention, repeat sequences of the target sequence are masked during the remove repeat step 200 by substitution of bases with Ns or Xs as described above and illustrated in FIG. 8. PPC table 315 includes fields to store data items representing an identifier for a primer pair 318, the forward sequence of the primer pair 320, the reverse sequence of the primer pair 322, the position of the forward sequence 324 on the target sequence, the position of the reverse sequence 326 on the target sequence, the melting temperature of the forward sequence 328 and the melting temperature of the reverse sequence. The PPC table can include fields to store other data items relating to the set of candidate primer pairs.

A primer pairs (PP) table 620 holds data item relating to a subset of substantially unique primer pairs from the set of all candidate primer pairs of the PPC table. The subset of unique primer pairs has had those primer pairs of the candidate set which are essentially duplicates of other primer pairs of the candidate set removed. PP table 620 includes fields to store data items corresponding to those in the PPC table, 621, 622, 623, 624, 625, 626 and 627 respectively, and supplemented by data items representing an identifier for a preceding primer pair 860 associated with a lowest cost route, a lowest cost value 628 associate with a primer pair and a selection flag 629 indicating whether the primer pair has been selected. The PP table can include fields to store other data items relating to the set of unique primer pairs.

A seed and bridges table 630 (GAP) has fields for holding data relating to a seed sequence used by the method and bridging sequences which are used as ‘fake’ sequences to bridge gaps in the reference sequence that are not covered by any of the candidate primer pairs. Fields are provided for a data item representing an identifier 632 for the seed or bridges, a data item representing a start position 634 on the target sequence associated with a seed or bridge sequence and a data item representing an end position 636 on the target sequence associated with a seed or bridge sequence.

A costs table 640 (EDGE) has fields for holding data items relating to the calculation of cost values (i.e. weightings) associated with the edge between a first primer pair and a second primer pair. Fields are provided for a data item representing an identifier for a first primer pair 642, an identifier for a second primer pair 644 and a cost 646 associated with the particular pair of primer pairs indicated by the primer pair identifiers.

A primers to add table 430 (PTA) has fields for holding data items relating to the ‘least cost path’ selected subset of primer pairs for amplifying the target sequence. The PTA table is used to store the results of the application of the single-source shortest-path algorithm. The PTA table includes fields for storing data items 431, 432, 433, 434, 436, 437 and 438 corresponding to the data items of the PPC and PP tables.

The removed sequence file 205 is a text file containing the target sequence with repeat sequences masked and is stored. The target sequence is typically between about 5 kilobases and 20 megabases in length.

FIG. 11 shows a flowchart 660 illustrating the execution of the computer program 602 which implements the method of selecting primer pairs and corresponds to step 400 as shown in FIGS. 1, 2 and 3. The candidate primer pairs file 315 output from the primer pair picking program is a text file containing primer pair reverse and forward sequences and associated information. The number of primer pairs present depends on the primer pair picking parameters used, and typically the file can include 10⁵ to 10⁶ primer pairs. The candidate primer pair file 315 is parsed 662 and the relevant data items are loaded into the PPC table 315 in the access database 606. The primer pairs are arranged in a sequentially ordered list in the PPC table, i.e. starting with the primer pair whose forward sequence is closest to the beginning of the target sequence and ending with the primer pair whose forward sequence is furthest from the beginning of the target sequence.

FIG. 12 is a schematic diagram illustrating the relationship between the reference sequence 902 and an illustrative candidate set of primer pairs A, A′, B, C, D, E, F, G and H. The reference sequence starts at position 904 and ends at position 906. Each primer pair is represented by an arrow extending from the start of the forward sequence of a primer pair to the end of the reverse sequence of that primer pair and directed from the beginning of the reference sequence toward the end of the reference sequence. For this candidate set of primer pairs, data for primer pair A is the first entry in table PPC and data for primer pair H is the last entry in table PPC. In this example, A, A′, B, C, D, E, F, G and H provide a unique identifier for each primer pair.

Routine 664 is used to remove similar primer pairs from the set of candidate primer pairs. FIG. 13A shows a flow chart 720 illustrating the routine for removing duplicate candidate primer pairs. In general, the set of candidate primer pairs is grouped into primer pairs covering the same part of the reference sequence and if there is more than one primer pair beginning and ending at the same position, then one of the primer pairs is retained and the rest are discarded.

The candidate primer pairs are arranged in the PPC table in sequential order. The candidate primer pairs are grouped into groups of primer pairs having forward sequences that start at the same position. The first group of candidate primer pairs is selected for evaluation 722 and the 5′ positions of the forward and reverse sequences of each of the primer pairs in the first group are compared 724. If it is determined 725 that there are duplicate primer pairs, i.e. a pair of primer pairs that start and end at the same positions, then one primer pair is retained the duplicate primer pairs are discarded 726. For example, as illustrated in FIG. 12, primer pairs A and A′ are duplicates and A′ is discarded. The next group of primer pairs along the reference sequence is then evaluated 727 and the process is repeated until all the groups of primer pairs have been evaluated along the reference sequence. After all the groups have been evaluated, then a unique set of candidate primer pairs results and their details are written from the PPC table to the PP table at step 728.

An optional step 665 can be carried out to further reduce the set of primer pairs, if there are sufficient primer pairs in the PP table that processing of the data is unlikely to be practicable. FIG. 13B shows a flow chart 730 illustrating this optional process. In general, the process involves binning the reference sequence at a fine scale, and identifying primer pairs whose forward reference sequence falls within the same bin. For such primer pairs, those having the longest and shortest amplicons are retained and the rest are discarded. This helps to reduce the data set while still providing a wide range of amplicon lengths for use in covering the reference sequence.

The reference sequence is binned into fifty base width bins starting from the beginning of the reference sequence to the end of the reference sequence. The first bin is selected 731 and those primer pairs whose forward sequence lies in the bin are identified 732 using data from the PPC table. The lengths of the amplicons for these primer pairs are determined 733 using the reverse sequence data from the PP table and the longest and shortest amplicons are selected 734 for retention. The remaining primer pairs are discarded. The PP table is then updated 735 so that the number of primer pairs having their forward sequence falling in the current bin has been reduced to two. The procedure is then repeated 736 for the next bin along the reference sequence, until the whole reference sequence has been evaluated. This procedure is optional and is used if it has been determined that it would be useful to further reduce the number of primer pairs after duplicates have been removed in order to allow processing of the data to be carried out in a reasonable time.

After the duplicate primer pairs have been removed from the candidate set, the program generates a seed 666. FIG. 14 shows a flow chart 740 illustrating the procedure for picking the seed sequence 910. Seed 910 is required in order to provide a starting point (vertex) for the cost calculation. The reference sequence 902 is defined by the position of a first base (position 1) 904 and the position of a last base (position n) 906 of a sequence of DNA. The seed picking procedure 740 starts by identifying 742 the position on the DNA sequence 5 bases prior to the start 904 of the reference sequence (‘−5 position’). Then the start position of the first primer pair A in table PP is determined 744. Then a base sequence from the −5 position up to the base immediately preceding the first base of the first primer pair A forward sequence is determined 746 as the seed sequence. The seed sequence 910 data is then written 748 into the GAP table 630.

After the seed has been picked the program finds any gaps 912 in the reference sequence not covered by primer pairs and determines bridging sequences to fill those gaps 666. FIG. 15 shows a flowchart 750 illustrating the procedure for picking bridges. Starting with the first primer pair A, its end position is determined 752. Then the start position of the next primer pair B in the table PP is determined. If the start position of next primer pair B is before the end position of the preceding primer pair A then they overlap and so there is no gap. If no gap is determined 756, then the current end position END is updated 758 to be equivalent to the end position of next primer pair B, provided that the end position of the next primer pair is greater than the end position of the current primer pair. It is then determined whether there are any more primer pairs in the table PP to be considered 760.

Primer pair B is now the nth primer pair and primer pair C is now the n+1th primer pair. The end position of primer pair B is determined or alternatively the current END value is used and the start position of primer pair C is determined and the procedure continues as above, and the END value is updated with the end of the C primer pair provided it is greater than the end position of the B primer pair. When a gap 912 is determined 756, e.g. between primer pairs C and D, then the program reads the base sequence of the reference sequence from the base adjacent the end of the nth primer pair C up to the base immediately preceding the first base of the n+1th primer pair D, and determines the start and end positions for this bridge sequence 762. The bridge data is then written 764 to the GAP table 630 and a bridge ID is generated and stored. The current END value is updated to the end position of the n+1th primer pair D and the procedure continues.

When it is determined 760 that the last primer pair in the PP table has been evaluated, then the sequence 914 of the reference sequence from the current END position to a position beyond the end of the reference sequence 906 determined and the GAP table 630 is updated 766 with the final bridge sequence data accordingly. A fixed position beyond the end of the current reference sequence is used so as to allow the program to accommodate reference sequences of greatly differing lengths, e.g. several orders of magnitude. The GAP table bridge sequence data is then added to the PP table data so that the PP table data contains a gapless sequence from the seed sequence all the way to the end of the reference sequence. The sequence from the end of the last primer pair H to beyond the end of the reference sequence becomes the last ‘primer pair’ in the PP table. The bridge sequences help to prevent primer pairs from being wrongly discriminated against during the cost evaluation as there are no primer pairs covering the gap sequence.

The program next calculates the costs 670 associated with every combination of sequential pairs of primer pairs in the PP table. FIG. 16 shows a flow chart 780 illustrating the procedure used in greater detail. The first primer pair in the list of primer pairs ordered by position in the PP table 620 is identified 782 and the next primer pair in the list is identified 784. In the first instance these will be the seed sequence and primer pair A respectively. Any gap between the end of the first primer pair (seed) and the start of the next primer pair (A) is determined and a gap cost is calculated 788 as the product of the length of any gap and a gap weighting factor Kg. In this embodiment Kg is set to ten. This gap cost penalizes primer pairs that do not overlap, thereby reducing the likelihood of the reference sequence being amplified fully. Then any overlap between the primer pairs is determined 790 and an overlap cost is calculated as the product of the length of any overlap and an overlap weighting factor Ko. In this embodiment Ko is set to one. This overlap cost penalizes primer pairs that overlap significantly, as the least number of primer pairs possible is preferred. Then an edge cost is calculated as the sum of the gap cost and the overlap cost and a fixed cost of adding another primer pair. In this embodiment, the ‘another primer pair’ weighting factor, is set at four thousand. The fixed cost of adding another primer pair penalizes having to use another primer pair to amplify the reference sequence as it is preferred to minimize the number of primer pairs.

It will be appreciated that it is the relative magnitude of the weighting factors which is important in assigning weightings to an edge, and that other sets of values of weighting factors can be used. Further, other costs and/or combinations of costs can be used in place of or to supplement the costs mentioned above. For example, the number of base pairs covered by a primer pair could be given a negative cost to reflect the benefit of covering more base pairs compared to a shorter primer pair. This could be implemented as a separate cost or alternatively the ‘add another primer pair’ cost could be made dependent on the primer pair coverage to reflect the number of base pairs covered (with pairs covering more base pairs having a lower ‘add another primer pair’ cost). The cost function could also take into account the properties of the primer pairs themselves relating to the amplification process. For example, a cost could be used which penalizes primer pairs having a melting temperature that is further from a reference melting temperature, such as the average melting temperature of the candidate primer pairs. Other costs could be used which reflect the suitably of a primer pair to be used with in the amplification reaction.

The ID for each of the primer pairs and the cost associated with the pair of primer pairs (Seed and A) are then written 796 to the EDGE table 640. It is then determined 798 if there are any more ‘next’ primer pairs in the ordered list and which are therefore toward the end of the reference sequence relative to the current primer pair (Seed). In this example, there are and the next primer pair (A) is updated 800 to be the next primer pair in the list which is B. The process is repeated and a cost associated with the seed and primer pair B is added to the EDGE table. This process is then continued until all the primer pairs in the list below Seed have been evaluated. When the cost for Seed and the last primer pair in the list (which is the end bridging sequence) have been evaluated, then the current primer pair is updated 804 so that the next primer pair in the list (A) becomes the current primer pair. Then the preceding steps are repeated for primer pair A and each primer pair in the list until the last primer pair has been evaluated. The procedure then stops when it is determined 802 that all costs for the last primer pair have been calculated. In this way the costs associated with passing from any one primer pair to another primer pair further down the reference sequence have been calculated and stored in the EDGE table 640 with identifiers for the pair of primer pairs.

The program then determines 672 the least cost between various sequential pairs of the primer pairs in table PP. FIG. 17 shows a flow chart 810 illustrating the procedure in greater detail. The first primer pair in the PP table is identified 812 and the next primer pair in the PP table is identified 814, which in the first instance are the Seed and A respectively. Next the lowest cost for every possible pair of primer pairs along the reference sequence is determined by searching the EDGE table 640. The cost from the current primer pair (Seed) to the next primer pair (A) is determined 816 by looking up the cost from the EDGE table 640. If the cost for the pair of primer pairs is determined 818 to be lower than the current lowest cost stored in the PP table, then the lowest cost is updated 820 in the PP table. In a first iteration there will be no lowest cost entry in the PP table and so the lowest cost is automatically updated with the cost for the pair.

It is then determined 822 if there are any remaining primer pairs in the list and if so the next primer pair is updated 824 to be the next primer pair in the list, which in this example is primer pair B. In this iteration step 816 has to look up the cost of the route Seed to B. The cost for this route is already stored in the EDGE table. Step 818 determines whether this routes has the lowest cost to get to B and if so, that cost is written 820 into the PP table for primer pair B together with the identifier for the preceding primer pair for that route to B. The next primer pair is then updated to C and step 816 has to calculate the costs of the route Seed to C. The PP table is updated with the lowest cost to get to C and the identifier for the preceding primer pair for that route to C and the process is continued until the lowest cost for all possible routes from Seed to the end of the reference sequence have been identified and written. As the end of the sequence is the last next primer pair for Seed, the current primer pair is updated at step 828 and A, as the next primer pair in the list to Seed is now the current primer pair. Then all routes from A to all primer pairs further down the reference sequence are evaluated and the lowest cost routes identified. For example, the route from A to B may be less costly than the route from Seed to B and so the PP table lowest cost entries are updated for B to reflect that the lowest cost route to B is actually from A and not from Seed. After all pairs of primer pairs starting with A have been evaluated, and the cost data items updated accordingly, the process proceeds to iterate the process for C, D, E, F, G and H to the end of the sequence have been calculated. The process therefore results in table PP having an identifier for the lowest cost to get to each of the primer pairs and the preceding primer pair involved in getting to each primer pair. For example the lowest cost route to G may be from E rather than from F and to the end of the sequence may be from H rather than from F or G.

The program now identifies 674 the least cost path and the primer pairs for that path. FIG. 19 shows a flow chart 830 illustrating the procedure for identifying the least cost path primer pairs. The ‘last primer pair’ is identified 832 and in the first iteration is the end sequence 914 between the end of the last primer pair H and beyond the end 906 of the reference sequence. The lowest cost data item for the last primer pair (end sequence) is read 834 from PP table together with the preceding primer pair corresponding to the lowest cost route to the end sequence. For example the lowest cost route to the end may have been H to end. The lowest cost data item is associated with the prior primer pair from which the lowest cost step to the end was made in the PP table. Therefore the primer pair involved in the last step of the route, H to End, is identified 836 and the PP table selected flag is set 838 for primer pair H indicating that H is part of the least cost route. It is then determined whether the seed has been reached yet 834.

If not then the last primer pair is updated to the prior primer pair, which in this example is H. Then the preceding primer pair field for H in the PP table is read 834, which identifies the previous primer pair involved in the lowest cost route to H. In this example coming from F may be identified as the lowest cost route and primer pair F is flagged as selected. Then the PP table entry for primer pair F is read and the primer pair involved in the lowest cost route to F is identified and the corresponding primer pair, E, is flagged as selected. The process is continued until the seed has been reached and then terminates. The end result is that the primer pair selection flags in the PP table indicates the best subset of primer pairs to be used in the amplification of the target sequence.

The computer program then removes 676 the seed, and bridging sequences from the PP table 620 and the primer pair data is added to the master database of primer pairs. FIG. 19 shows a flowchart 850 illustrating the results output processes of the computer program in greater detail. The IDs for the flagged primer pairs are written into the ID field of the primers to add table 852 and then the related primer pair data is added 854 from the primer pair table. The data in the PTA table is then added 856 to the master database of primer pairs. The method for selecting a subset of primer pairs from the candidate set of primer pairs is then completed.

Amplification Reaction

The amplification reaction involves both an amplification reaction mix or cocktail and thermocycling parameters. In one application of the present invention, the reaction mix was prepared by making two master reaction mixes, then adding an aliquot of each mix to the primer pairs in the following manner:

PCR Set Up:

11.68 μL total volume reactions

| | Volume | | |
|---|---|---|---|
| Reagents: | per reaction | | Final Amount per reaction |
| Water | 4.575 | μL | |
| dNTPs, 10 mM (0.39 μL) | 1.56 | μL | 334 μM each |
| each | | | |
| template DNA (100 ng/μL) | 0.145 | μL | 14.5 ng |
| 10% DMSO/5 M betaine | .49 | μL | 0.42%/0.21 M |
| 140 mM NH₄SO₄/500 mM | 1.077 | μL | 12.9 mM/46 mM |
| Tris | | | |
| 25 mM MgCl₂ | 0.172 | μL | 368 μM |
| Taq Polymerase (2.5 U/μL) | 0.23 | μL | 0.58 units |
| Taq antibody | 0.184 | μL | 0.2 μg |
| 50 mM KCl/10 mM Tris-HCl | 0.627 | μL | 2.7 mM/0.54 mM |
| DMSO | 0.34 | μL | 2.9% |
| Tricine (1M) | 0.28 | μL | 24 mM |
| Total Volume: | 9.68 | μL | |

Each 9.68 μl reaction mix was added to tubes containing 2 μl of a pair of primers (a forward primer and a reverse primer), for a final concentration of 192 nM each primer in a final 11.68 μL reaction volume. The reaction cocktails were then used to run amplification reactions as described infra.

In an alternative embodiment of the present invention, the taq polymerase can be eliminated, and instead combined with 0.015 μg/μL TaqStart antibody and buffer to form an antibody-bound taq complex which is then added to the reaction cocktail.

Reagents for the reaction cocktails can be obtained from the following sources: dNTP's (Life Technologies), Taq polymerase (Roche Molecular Biosciences, Epicentre Techno logies, Biorad Laboratories or Applied Biosystems), tricine, tris, NH₄SO₄, MgCl₂, betaine, and DMSO (Sigma Aldrich), Taqstart antibody (Clontech).

In one example, the cycling conditions were as follows:

Initial heating step: 95° C. for 3 minutes

10 cycles of:

heating step: 94° C. for 2 seconds

cooling step: 64° C. for 15 minutes

28 cycles of:

heating step: 94° C. for 2 seconds

cooling step: 64° C. for 15 minutes for the first cycle, with an increase in time of 20 seconds in each subsequent cycle

Final cooling step: 62° C. for 60 minutes

4° C. hold

Also, in an alternative example of the present invention, the cycling conditions were as follows:

Initial heating step: 94° C. 3 minutes

35 cycles of:

heating step: 94° C. for 2 seconds

cooling step: 62° C. for 12 minutes

Final cooling step: 62° C. for 25 minutes

4° C. hold

Aliquots of each completed amplification reaction were run on a 0.8% agarose gel and visualized with ethidium bromide.

The above description is illustrative and not restrictive. Many variations of the invention will become apparent to those of skill in the art upon review of this disclosure. The scope of the invention should, therefore, be determined with reference to the appended claims along with their full scope of equivalents.

[image]

[image]

[image]

[image]

[image]

[image]

[image]

[image]

[image]

[image]

[image]

[image]

[image]

What is claimed is:

1. A method for selecting primer pairs for amplifying a target sequence, comprising the steps of:
choosing a reference sequence;
masking at least selected repeat regions in said reference sequence to yield a masked reference sequence;
selecting primer sequences from said masked reference sequence to yield a set of primers;
evaluating said set of primers for extent of coverage and overlap of said masked reference sequence; and
selecting a subset of primer pairs having reduced overlap from said set of primers.

2. The method of claim 1, wherein said primer sequences are selected according to two or more parameters including primer length and primer melting temperature.

3. The method of claim 1, wherein said step of selecting a subset of primer pairs selects a subset of primer pairs with a minimal or substantially minimal number of primer pairs required to amplify said target sequence.

4. The method of claim 3, wherein said step of selecting a subset of primer pairs selects a subset of primer pairs with a least number of primer pairs required to amplify said target sequence.

5. The method of claim 3, wherein said second selecting step selects said subset of primer pairs according to at least one parameter selected from the group of overlap length, gaps between pairs of primer pairs, and necessity of adding another primer pair to the subset.

6. The method of claim 1, wherein said step of selecting a subset of primer pairs is performed by a computer program and said computer program executes a single-source shortest-path algorithm to select said subset of primer pairs.

7. The method of claim 1, wherein said step of selecting a subset of primer pairs is performed by a computer program and said computer program executes an algorithm solving a single-source shortest path problem on a weighted, directed graph G=(V,E) for the case in which all edge weights are normegative, and w(u,w)≧0 for each edge (u,v)εE.

8. The method of claim 1, wherein said target sequence is genomic DNA from a human species.

9. The method of claim 1, wherein said target sequence is genomic DNA from a non-human primate species.

10. The method of claim 1, wherein said reference sequence is genomic DNA from a human species.

11. A computer program for selecting primer pairs for amplifying a target nucleic acid sequence comprising:
computer code that receives input of a reference sequence;
computer code that masks at least selected repeat regions in said reference sequence to yield a masked reference sequence;
computer code that selects primer sequences from said masked reference sequence to yield a set of primers;
computer code that evaluates said set of primers for extent of coverage and overlap of said masked reference sequence; and
computer code that selects a subset of primer pairs having reduced overlap from said set of primers.

12. The computer program of claim 11, wherein said primer sequences are selected according to two or more parameters including primer length and primer melting temperature.

13. The computer program of claim 11, wherein said computer code executes an algorithm that in said second selecting step selects a subset of primer pairs with a minimal or substantially minimal number of primer pairs required to amplify said target sequence.

14. The computer program of claim 11, wherein said computer code executes an algorithm that in second selecting step selects said subset of primer pairs according to at least one parameter selected from the group of overlap length, gaps between pairs of primer pairs, and necessity of adding another primer pair to the subset.

15. The computer program of claim 11, wherein said computer code executes a single-source shortest-path algorithm.

16. A system that selects primer pairs for amplifying a target nucleic acid sequence comprising:
a processor; and
a computer readable medium coupled to said processor for storing a computer program comprising:
computer code that receives input of a reference sequence;
computer code that masks at least selected repeat regions in said reference sequence to yield a masked reference sequence;
computer code that selects primer sequences from said masked reference sequence to yield a set of primers;
computer code that evaluates said set of primers for extent of coverage and overlap of said reference sequence; and
computer code that selects a subset of primer pairs having reduced overlap from said set of primers.

17. The system as claimed in claim 16, wherein the computer code selects primer sequences according to two or more parameters including primer length and primer melting temperature.

18. A method for selecting a subset of primer pairs from a set of candidate primer pairs for amplifying a target nucleic acid sequence, comprising:
providing a reference sequence;
evaluating said set of candidate primer pairs by scoring the usefulness in amplifying the reference sequence of primer pairs from the candidate set of primer pairs to identify a subset of primer pairs; and
selecting the subset of primer pairs from said set of candidate primer pairs.

19. The method of claim 18, wherein evaluating said set of candidate primer pairs includes determining the extent of any overlap at least one pair of primer pairs from said set of candidate primer pairs.

20. The method of claim 18, wherein evaluating said set of candidate primer pairs includes determining the extent of any gap between at least one pair of primer pairs from said set of candidate primer pairs.

21. The method of claim 18, wherein evaluating said set of candidate primer pairs includes considering the total number of primer pairs in the subset.

22. The method of claim 18, wherein evaluating the set of candidate primer pairs includes minimizing the number of primer pairs in the subset.

23. The method of claim 18, wherein evaluating the set of candidate primer pairs includes applying a single-source, shortest-path algorithm to the candidate set of primer pairs.

24. The method of claim 18, including removing similar primer pairs from the candidate set of primer pairs.

25. The method of claim 18, wherein said reference sequence has been masked to remove at least some repeat sequences of said target sequence.

26. The method of claim 18, wherein evaluating said set of candidate primer pairs includes assigning a cost to a primer pair from the set of candidate primer pairs reflecting the suitability of the primer pair for use in amplifying the target sequence.

27. A method for amplifying a target sequence, comprising the steps of:
mixing a reaction cocktail comprising deoxynucleotide triphosphates, target DNA, a divalent cation, DNA polymerase enzyme, a broad spectrum solvent, a zwitterionic buffer and at least one primer pair having a length of about 28 nucleotides to about 36 nucleotides and a melting temperature of about 72° C. to about 88° C.;
heating said reaction cocktail at a denaturing temperature of about 90° C. to about 96° C. for about 1 second to about 30 seconds;
cooling said reaction cocktail at an annealing/extension temperature of about 50° C. to about 68° C. for about 1 minute to about 28 minutes;
repeating said heating and cooling steps at least 10 times; and
cooling said reaction cocktail to 4° C. in a final cooling step.

28. The method of claim 27, wherein said reaction cocktail comprises about 50 μM to about 400 μM of each primer of said at least one primer pair, about 200 μM to about 500 μM each dNTP, about 0.02 ng/μl to about 2.5 ng/μl template (target) DNA, 0.0% to about 7.0% broad spectrum solvent, 0.0 M to about 0.75 M betaine, about 7 mM to about 35 mM NH₄SO₄, about 25 mM Tris to about 125 mM Tris, about 100 μM to about 500 μM MgCl₂, about 0.01 units/μl to about 0.20 units/μl polymerase, and 0 mM to about 50 mM zwitterionic buffer.

29. The method of claim 28, wherein said reaction cocktail comprises about 100 nM to about 240 nM of each primer of said at least one primer pair, about 300 μM to about 400 μM each dNTP, about 0.05 ng/μl to about 1.5 ng/μl template (target) DNA, 1.5% to about 4.5% broad spectrum solvent, 0.2 M to about 0.6 M betaine, about 10 mM to about 20 mM NH₄SO₄, about 40 mM Tris to about 80 mM Tris, about 250 μM to about 400 μM MgCl₂, about 0.025 units/μl to about 0.07 units/μl polymerase, and 10 mM to about 30 mM zwitterionic buffer.

30. The method of claim 29, wherein said reaction cocktail comprises about 192 nM of each primer of said at least one primer pair, about 385 μM each dNTP, about 1.2 ng/μl template (target) DNA, about 3.7% DMSO, about 0.24 M betaine, about 13 mM NH₄SO₄, about 48 mM Tris, about 385 μM MgCl₂, about 0.05 units/μl polymerase, and 25 mM Tricine.

31. The method of claim 27, wherein a duration of each of said cooling step increases during the repeating step.

32. The method of claim 27, wherein said reaction cocktail further comprises about 0.005 μg/μl to about 0.10 μg/μl taq antibody.

33. The method of claim 27, wherein an initial heating step is performed before said heating step.

34. The method of claim 27, wherein an additional cooling step is performed after said repeating step and before said final cooling step.
